(19) United States
(12) Patent Application Publication
(10) Pub. No.: US 2011/0135575 A1
(43) Pub. Date: Jun. 9, 2011

(54) METALLOPROTEIN MRI CONTRAST AGENTS AND RELATED METHODS

(75) Inventors: Mikhail G. Shapiro, Boston, MA (US); Philip Romero, Pasadena (CA); Robert S. Langer, Newton, MA (US); Frances Arnold, La Canda Flintridge (CA); Alan Preadip Jasanoff, Combridge, MA (US)

(73) Assignee: Massachusetts Institute of Technology, Cambridge, MA (US)

(21) Appl. No.: 12/675,256

(22) PCT Filed: Aug. 28, 2008
(86) PCT No.: PCT/US08/10198

§ 371 Date: Feb. 24, 2011

Related U.S. Application Data
(60) Provisional application No. 60/966,568, filed on Aug. 28, 2007.

(51) Int. Cl. A61B 5/055; G01N 24/08; G01N 33/20; G01N 33/68; C12Q 1/26; C07H 21/00; C12N 15/63; C12N 5/00; C07K 2/00

(52) U.S. Cl. 424/9.3; 436/173; 436/73; 436/86; 436/98; 436/129; 436/111; 436/81; 436/96; 436/89; 436/94; 435/25; 536/23.1; 435/320.1; 435/325; 530/300

(57) ABSTRACT

The present invention generally relates to Magnetic Resonance Imaging (MRI) analysis of the location, presence, and/or quantity of a particular molecule or material. In some embodiments, the invention relates to compositions and methods for determination of an analyte, wherein a compositions may produce an MRI signal that can be affected by the presence of a particular composition (e.g., analyte). In a particular embodiment, the composition comprises a protein. The invention also provides related nucleic acid molecules, polypeptides, and fragments thereof. Compositions of the invention may exhibit reduced toxicity and may be readily delivered in vivo. Various embodiments of the invention may be useful as sensors, diagnostic tools, and the like.

### FIELD OF THE INVENTION

The present invention relates to compositions and methods for magnetic resonance imaging (MRI) and/or determination of analytes.

### BACKGROUND OF THE INVENTION

Molecular imaging is a rapidly expanding field with great market potential, as evidenced by recent acquisitions of molecular imaging companies by large multinational corporations such as GE, and the success of startup companies like Quantum Dot Corp. In the MRI field, FDA approved contrast agents such as gadolinium chelates (e.g. Resovist) and superparamagnetic nanoparticles (e.g. MION) are already in wide clinical use for vascular-related imaging.

Many current molecular sensors for MRI involve either large synthetic organic compounds or collections of inorganic metal oxide nanoparticles. Due to their synthetic nature, these sensors possess significant disadvantages, including the need to perform a novel chemical synthesis for each new sensor design, which can be especially arduous and can require many synthetic iterations before producing a sensor. In addition, synthetic contrast agents can be difficult to deliver in vivo, and delivery modes and toxicity levels need to be addressed on an individual basis by modifying the chemistry and formulation of each sensor.

Accordingly, improved compositions and methods are needed.

### SUMMARY OF THE INVENTION

The present invention provides methods for determining an analyte comprising introducing a genetically-engineered protein molecule into a biological sample and, if an analyte is present, allowing the analyte to bind to the genetically-engineered protein molecule; and exposing the sample to magnetic resonance imaging conditions, thereby determining the presence and/or amount of the analyte in the biological sample.

The present invention also provides methods for determining an analyte comprising providing a genetically-engineered protein molecule having a first relaxivity upon exposure to magnetic resonance imaging conditions; exposing the genetically-engineered protein molecule to a biological sample suspected of containing an analyte, wherein the genetically-engineered protein molecule interacts with the analyte, if present, to generate a second relaxivity of the genetically-engineered protein molecule, upon exposure to said magnetic resonance imaging conditions; and determining a change, or lack thereof, between the first and second relaxivities, thereby determining the presence and/or amount of the analyte in the biological sample.

The present invention provides methods for determining an analyte comprising providing a protein molecule having a determinable magnetic resonance imaging signal upon exposure to magnetic resonance imaging conditions, wherein the protein is not hemoglobin or myoglobin; exposing the protein molecule to a biological sample suspected of containing an analyte, wherein the protein molecule interacts with the analyte, if present, to generate an analyte-bound protein magnetic resonance imaging signal, upon exposure to said magnetic resonance imaging conditions, that is shifted relative to the protein magnetic resonance imaging signal absent the analyte; and determining the shift in the magnetic resonance imaging signal, or lack thereof, thereby determining the presence and/or amount of the analyte in the biological sample.

The present invention provides methods for determining an analyte comprising providing a protein molecule having a determinable magnetic resonance imaging signal upon exposure to magnetic resonance imaging conditions; exposing the protein molecule to a biological sample suspected of containing an organic analyte, wherein the protein molecule interacts with the organic analyte, if present, to generate an analyte-bound protein magnetic resonance imaging signal, upon exposure to said magnetic resonance imaging conditions, that is shifted relative to the protein magnetic resonance imaging signal absent the analyte; and determining the shift in the magnetic resonance imaging signal, or lack thereof, thereby determining the presence and/or amount of the organic analyte in the biological sample.

The present invention provides methods for determining an analyte comprising providing a magnetic resonance imaging contrast agent sensor having a relaxivity which, in the presence of an analyte, undergoes a shift in relaxivity of at least 0.1 mM⁻¹ s⁻¹; introducing the contrast agent sensor into a biological sample; and determining the shift in relaxivity of at least 0.1 mM⁻¹ s⁻¹, or lack thereof, thereby determining the presence and/or amount of the analyte in the biological sample.

The present invention also relates to isolated nucleic acid molecules selected from the group consisting of: (a) complements of nucleic acid molecules that hybridize under high stringency conditions to a second nucleic acid molecule comprising a nucleotide sequence set forth as any of the nucleotide sequences in FIG. 31, (b) nucleic acid molecules that differ from the nucleic acid molecules of (a) in codon sequence due to the degeneracy of the genetic code, and (c) full-length complements of (a) or (b).

The present invention also relates to isolated nucleic acid molecules that comprise

one or more nucleotide sequences as set forth in FIG. 31, or full-length complements thereof.

The present invention also relates to isolated nucleic acid molecules comprising a nucleotide sequence that is at least about 90% identical to a nucleotide sequence set forth as any of the nucleotide sequences in FIG. 31, or a full-length complement thereof.

The present invention also relates to compositions comprising any isolated nucleic acid molecule described herein, and a carrier. The present invention also relates expression vectors comprising any isolated nucleic acid molecule described herein operably linked to a promoter.

The present invention also relates to isolated host cells transformed or transfected with any expression vector described herein. The present invention also relates to compositions comprising isolated host cells described herein, and a carrier.

The present invention also relates to isolated polypeptides encoded by isolated nucleic acid molecules described herein, or a fragment thereof that is at least eight amino acids in length.

The present invention also relates to compositions comprising isolated polypeptides described herein, and a carrier.

The present invention also provides kits comprising one or more nucleic acid molecules that hybridize under high stringency conditions to a nucleotide sequence set forth as any of the nucleotide sequences in FIG. 31.

### BRIEF DESCRIPTION OF THE DRAWINGS

FIG. 1 shows schematic representation of a protein-based sensor and its use in determining an analyte, according to one embodiment of the invention.

FIG. 2 shows an MRI image of a microplate acquired using a T1-weighted acquisition sequence, wherein the microplate contains various mutant BM3 proteins upon exposure to arachidonic acid.

FIG. 3A shows a graph of T₁ relaxivity (r₁), as well as contrast images, of BM3h in (i) PBS, (ii) AA, and (iii) DA.

FIG. 3B shows a graph of T₁ relaxation rates (1/T₁) measured from solutions of BM3h incubated with AA.

FIG. 3C shows optical absorbance spectra of BM3h under various conditions.

FIG. 3D shows difference spectra showing the change in BM3h absorbance as a function of wavelength under various conditions.

FIG. 3E shows normalized titration curves showing binding of BM3h to AA or DA.

FIG. 4A shows the absorbance spectra of an evolved BM3 protein upon exposure to (i) 0 mM arachidonic acid and (ii) 1 mM arachidonic acid.

FIG. 4B shows the corresponding absorbance difference spectrum for the absorbance spectra in FIG. 4A.

FIG. 5A shows the absorbance spectra of the mutant BM3 protein (C2B12H) upon exposure to (i) 0 mM chlorzoxazone and (ii) 2 mM chlorzoxazone

FIG. 5B shows the corresponding absorbance difference spectrum for the absorbance spectra in FIG. 5A.

FIG. 6A shows the absorbance spectra of the mutant BM3 protein upon exposure to (i) 0 mM seratonin and (ii) 2 mM seratonin.

FIG. 6B shows the corresponding absorbance difference spectrum for the absorbance spectra in FIG. 6A.

FIG. 7A shows the absorbance spectra of the mutant BM3 protein upon exposure to (i) 0 mM dopamine and (ii) 2 mM dopamine, and FIG. 7B shows the corresponding absorbance difference spectrum.

FIG. 7B shows the corresponding absorbance difference spectrum for the absorbance spectra in FIG. 7A.

FIG. 8 shows the T1-weighted images for the wild type BM3 (WT) and mutant BM3 (5H6) proteins in the presence of (i) water, (ii) seratonin (5HT), (iii) dopamine (DA), and (iv) melatonin (Mel or MT).

FIG. 9 shows the T1 relaxivities for the wild type BM3 (WT) and mutant BM3 (5H6) proteins in the presence of (i) water, (ii) seratonin (5HT), (iii) dopamine (DA), and (iv) melatonin (Mel or MT).

FIG. 10A shows a plot of relaxivity values measured from (i) BM3h-B7 in PBS, (ii) BM3h-B7 in DA, (iii) BM3h-8C8 in PBS, and (iv) BM3h-8C8 in DA.

FIG. 10B shows T₁-weighted MRI contrast images of (i) BM3h-B7 in PBS, (ii) BM3h-B7 in DA, (iii) BM3h-8C8 in PBS, and (iv) BM3h-8C8 in DA.

FIG. 10C shows a normalized MRI image showing signal amplitudes measured from wells containing WT BM3h, BM3h-8C8, and BM3h-B7, each incubated with increasing DA concentrations.

FIG. 10D shows a graph of relaxation rate (1/T₁ values) measured from solutions of (i) WT, (ii) BM3h-B7, and (iii) BM3h-8C8, as a function of total DA concentration.

FIG. 10E shows a graph of 1/T₁ relative to ligand-free protein for BM3h-B7 (gray bars) and BM3h-8C8 (black bars) incubated with various substrates.

FIG. 10F shows a graph of the affinities of BM3h-B7 and BM3h-8C8 for DA, 5HT, and norepinephrine (NE), as spectroscopically determined.

FIG. 11 shows graphs of the optical density of DA binding to (a) BM3h-B7 and (b) BM3h-8C8.

FIG. 12 shows (a) absorbance spectra of BM3h-B7 at various filtration steps and (b) a plot of the absorbance of BM3h-B7 from 430 nm to 410 nm in the absence (white bars) and presence (gray bars) of DA at various filtration steps, (c) absorbance spectra of BM3h-8C8 at various filtration steps and (b) a plot of the absorbance of BM3h-8C8 from 430 nm to 410 nm in the absence (white bars) and presence (gray bars) of DA at various filtration steps.

FIG. 13A shows the absorption spectra of a wild type BM3 protein with increasing amounts of dopamine as the analyte.

FIG. 13B shows a plot of the calculated change in absorbance upon each addition of analyte.

FIG. 13C shows the plot of the distance between the maximum and minimum points on each difference spectrum as a function of analyte concentration, generating a binding isotherm.

FIG. 14 show the spectroscopic titration curve for a wild type BM3 protein in the presence of (i) dopamine, (ii) seratonin, and (iii) chlorzoxazone.

FIG. 15 shows the spectroscopic titration curve for a mutant BM3 protein (5H6) in the presence of (i) dopamine, (ii) seratonin, and (iii) chlorzoxazone.

FIG. 16 shows a graph of the differences in baseline (analyte-free) T1 relaxivity for a series of mutant BM3 proteins.

FIG. 17A shows a schematic representation of a directed evolution approach, according to one embodiment of the invention.

FIG. 17B shows histograms of mutant DA dissociation constants determined during five rounds of directed evolution.

FIG. 17C shows a graph of dissociation constants for DA and AA.

FIG. 17D shows titration curves of DA binding to WT BM3h and proteins selected after various rounds of directed evolution.

FIG. 17E shows an X-ray crystal structure of WT BM3h bound to palmitoleic acid.

FIG. 18 shows the absorbance difference spectra collected sequentially from a single well of a 96-well plate containing lysates of mutant library as increasing amounts of ligand are added to the well.

FIG. 19 shows a curve-fitted plot estimating a dissociation constant between an analyte and a mutant protein, based on the absorbance spectra in FIG. 18.

FIG. 20 shows sample plots for the “fold increase” and “fold decrease” for mutants screened against arachidonic acid and dopamine, respectively.

FIG. 21 shows bulk titration curves for the following BM3 proteins and mutants in the presence of increasing amounts of dopamine: (i) wild type, (ii) EPC38, (iii) C88A12, (iv) C87C9, (v) C89D11, (vi) C87E7, (vii) C89B1, and (viii) C810H10.

FIG. 22 shows a graph of the (i) ligand-free (e.g., water, 0 mM analyte) and (ii) ligand-saturated (e.g., 1 mM dopamine) relaxivity for various mutant proteins.

FIG. 23 shows a plot of the dopamine Kd for the wild type heme domain of cytochrome P450-BM3 and the mutant selected from four rounds of evolution.

FIG. 24 shows the dopamine titration curves for the following BM3 proteins and mutants: (i) wild type, (ii) EP3C8, (iii) C810H10, (iv) H103D8, and (v) D88C8.

FIG. 25 shows a plot of the T1 relaxation rates at various concentrations of a mutant protein selected after round 4 of directed evolution (“8C8”) in phosphate buffered saline measured at 4.7 T, (i) without dopamine and (ii) with 1 mM dopamine.

FIG. 26 shows a T1-weighted MRI image of sample wells containing a constant concentration of 8C8, with varying concentrations of dopamine ranging from 0 to 200 uM.

FIG. 27 shows amino acid sequences of the wild-type heme domain of cytochrome P450-BM3 (WT) and that of 8C8.

FIG. 28A shows a schematic representation of stimulation of PC12 cells to release DA into supernatants containing a BM3h-based sensor.

FIG. 28B shows a plot of T₁-weighted spin echo MRI signal amplitudes measured from the supernatants of PC12 cells incubated with BM3h-B7.

FIG. 28C shows a plot of relaxation rates measured from samples minus the relaxation rate of buffer not containing BM3h-based sensors.

FIG. 28D shows a plot of estimated concentrations of DA in various samples.

FIG. 29 shows a plot of T1 relaxation rates of the supernatant of cells resuspended in high-potassium and low-potassium conditions.

FIG. 30 shows a graph of DA release from PC12 cells by BM3h-8C8 in the presence of various ions.

FIG. 31 shows original and optimized coding sequences for wild type BM3 heme domain.

FIG. 32A shows a schematic representation of paired injections into the striatum of craniotomized rats.

FIG. 32B shows MRI images of a rat brain.

FIG. 32C shows a plot of MRI signal changes as a function of time for BM3h-8C8 in the presence and absence of DA.

FIG. 32D shows a plot of MRI signal changes as a function of time for WT BM3h in the presence and absence of DA.

FIG. 32E shows a plot of MRI signal changes as a function of time for BM3h-8C8 in the presence of DA and DA+Coc.

Other aspects, embodiments and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

The present invention generally relates to Magnetic Resonance Imaging (MRI) and/or optical analysis of the location, presence, and/or quantity of a particular molecule or material. In particular, MRI imaging agents are provided that are detectable in an MRI apparatus, i.e., they are contrast agents that provide information for producing an MRI image, and an optical, MRI, or other signal produced by the agent may be affected by the presence of a particular composition (e.g., analyte). Accordingly, in one aspect, the present invention relates to compositions and methods for determination of an analyte. The invention further relates to nucleic acid molecules, polypeptides, and fragments thereof. Various embodiments of the invention may be useful as sensors, diagnostic tools, and the like.

In some embodiments, the invention provides methods for determination of analyte, wherein the analyte may be determined by monitoring, for example, a change in an optical or MRI signal of a species (e.g., protein) upon exposure to an analyte. In some cases, the present invention may be used for the detection of neurotransmitters. The present invention also provides various compositions for determination of analyte. In some embodiments, the compositions may be readily synthesized and/or modified using one or more genetic engineering methods in order to suit a particular application, i.e., determination of a particular analyte. Compositions of the invention may exhibit reduced toxicity and may be readily delivered in vivo. The present invention also allows for the selection and design of sensor compositions, independent of delivery method.

Methods for the determination of analytes may comprise exposure of a species (e.g., a protein) to a sample suspected of containing the analyte, and, if present, the analyte interacts with the species to cause a change in a signal of the species (e.g., an optical signal, a relaxivity). In some embodiments, the analyte may interact with the species to cause a change in a magnetic resonance imaging signal of the species, which may then determine the analyte. For example, the species may be a polypeptide (e.g., protein) molecule having an MRI signal, wherein interaction of the polypeptide with an analyte generates a determinable change or shift in the MRI signal, thereby indicating the presence and/or amount of the analyte.

In some cases, the change in signal comprises a shift in the absorbance spectrum of the species. In some cases, the change comprises a shift in a relaxivity (e.g., T1, T2, etc.) of the species. In some embodiments, the change may be a visible color change. As used herein, the term “determining” generally refers to the analysis of a species or signal, for example, quantitatively or qualitatively, and/or the detection of the presence or absence of the species or signals. “Determining” may also refer to the analysis of an interaction between two or more species or signals, for example, quantitatively or qualitatively, and/or by detecting the presence or absence of the interaction. The compositions and methods described herein may be incorporated into devices such as sensors or any device or article capable of detecting an analyte. In some embodiments, the analyte (e.g., dopamine, seratonin) may be determined when present in a subject (e.g., human). Some embodiments of the invention provide methods for non-invasively determining (e.g., imaging) the presence and/or amount (e.g., concentration) of analytes such as neurotransmitters within living tissues.

Methods of the invention may comprise introduction of a species into a biological sample suspected of containing an analyte and, if an analyte is present, allowing the analyte to bind to the species. In some embodiments, the analyte may bind to a metal ion associated with the species, optionally replacing or substituting a ligand of the species. In the absence of analyte in the biological sample and/or prior to binding of the analyte, the species may have a first determinable signal, and, upon binding of the analyte to form an analyte-bound species, a second determinable signal may be generated that is changed or shifted relative to the first signal. The biological sample may then be exposed to magnetic resonance imaging conditions, thereby determining the presence and/or amount of the analyte in the biological sample. In some cases, determination of the analyte is performed in vivo. In some cases, determination of the analyte is performed in vitro.

The determinable signal may comprise a magnetic resonance imaging (MRI) signal. For example, the species may be a paramagnetic species having a first relaxivity upon exposure to magnetic resonance imaging conditions. Exposure of the species to a biological sample suspected of containing an analyte, wherein the species interacts with (e.g., binds) the analyte, if present, may then generate a second relaxivity of the analyte-bound species upon exposure to said magnetic resonance imaging conditions. Determination of a change, or lack thereof, between the first and second relaxivities may then determine the presence and/or amount of the analyte in the biological sample. In some embodiments, the relaxivity may be a T1 relaxivity. In some embodiments, the relaxivity may be a T2 relaxivity.

As used herein, “magnetic resonance imaging conditions” refers to a set of conditions under which a species may generate a magnetic resonance imaging signal. For example, a species comprising a paramagnetic metal ion may be capable of producing an MRI signal having either longitudinal or transverse proton relaxation enhancement, when exposed to MRI conditions. The set of conditions typically comprises exposure to a magnetic field, wherein the resulting MRI signal may be determined by observation of the relaxivity of the species. The MRI signal may also be used to produce an MRI image, wherein the contrast between locations which comprise the species and locations which do not comprise the species may be used to form the image. In some cases, the image may be formed based on the contrast between locations wherein an analyte is present and has interacted with the species, and locations wherein an analyte is not present or is present in a different amount. Those of ordinary skill in the art would understand the use of MRI signals to produce an MRI contrast image.

As described herein, determination of an analyte may involve determination of a change or shift in MRI signal of a species comprising a paramagnetic metal ion upon binding of the analyte to the paramagnetic metal ion, upon exposure to magnetic resonance imaging conditions. Without wishing to be bound by theory, the binding of the analyte to the paramagnetic metal ion can affect the magnitude or type of MRI signal produced by the metal ion. The mechanism of such effect may involve, for example, a change in the accessibility of the metal ion to coordination by other ligands such as water, or a change in the metal ion's spin state. In some cases, the change in the MRI signal comprises a change in the contrast produced by the species in an MRI image.

The term “paramagnetic metal ion” is known in the art and refers to a metal ion having unpaired electrons, causing the metal ion to have a measurable magnetic moment in the presence of an externally applied field. Examples of suitable paramagnetic metal ions, include, but are not limited to, ions of iron, nickel, manganese, copper, gadolinium, dysprosium, europium, and the like. For example, Gd(III), Fe(III), Mn(II), Yt(III), Dy(III), and Cr(III) are examples of paramagnetic metal ions. In some embodiments, a protein of the invention, such as a genetically-engineered protein of the invention, comprises a paramagnetic metal ion, wherein the paramagnetic ion is an ion of iron, nickel, manganese, copper, gadolinium, dysprosium, or europium. In some cases, the paramagnetic metal ion may be an ion of iron, nickel or manganese.

Some embodiments of the invention may comprise a shift in relaxivity of at least 0.1 mM⁻¹ s⁻¹, upon binding of an analyte to the species (e.g., a protein). In some embodiments, the shift in relaxivity may be at least 0.25, 0.50, 0.75, 1.0, 2.5, 5.0, 7.5, 10.0 mM⁻¹ s⁻¹, or greater, upon binding of an analyte to the species. In some cases, methods of the invention comprise determining a change in an optical signal of the species, such as the wavelength emitted or absorbed the MRI agent. For example, the interaction between the analyte and a species (e.g., protein molecule) may cause a shift in an absorption and/or emission wavelength characteristic of the MRI agent. In some cases, the change comprises a blue-shifted change in the wavelength of the absorption and/or emission of the MRI agent, i.e., the absorption and/or emission may be shifted to a shorter wavelength. In some cases, the change comprises a red-shifted change in the wavelength of the absorption and/or emission of the MRI agent, i.e., the absorption and/or emission may be shifted to a longer wavelength. The wavelength of the absorption and/or emission of the MRI agent in the presence of analyte may be separated from the absorption and/or emission of the MRI agent in the absence of analyte by at least 5 nm, 10 nm, 25 nm, at least 50 nm, at least 75 nm, at least 100 nm, at least 150 nm, or greater. The wavelength of an absorption and/or emission refers to the wavelength at which the peak maximum of the absorption and/or emission occurs in an absorbance and/or emission spectrum. The absorption and/or emission may be a particular peak having the largest intensity in an absorption and/or emission spectrum, or, alternatively, the absorption and/or emission may be a peak in an absorption and/or emission spectrum that has at least a defined maximum, but has a smaller intensity relative to other peaks in the absorption and/or emission spectrum.

In some embodiments, the change in absorption and/or emission may occur for an MRI agent with substantially no shift in the wavelength of the absorption and/or emission, wherein a peak maximum of the absorption and/or emission changes (e.g., increases or decreases) but the wavelength remains essentially unchanged. In other embodiments, the change in peak maximum may occur for the absorption and/or emission of an MRI agent in combination with a shift in the wavelength of the absorption and/or emission. For example, the absorption and/or emission of an MRI agent may simultaneously undergo a shift in wavelength in addition to an increase or decrease in peak maximum.

In some cases, the change in the absorption and/or emission of may also be visible by sight, e.g., the protein may emit visible light. This may allow for the determination of analytes via a colorimetric change. For example, a metalloprotein, in the absence of analyte, may have a first color, and, upon exposure to an analyte and irradiation by a source of energy, the metalloprotein may have a second color, wherein the change in color may determine the analyte.

In some embodiments, the species capable of interacting with (e.g., binding) the analyte may be a biological molecule, such as a protein. Protein that may be suitable for use in the context of the invention may comprise at least one paramagnetic metal ion (e.g., a metalloprotein), as well as at least one binding site for an analyte of interest. The paramagnetic metal ion may be positioned within or adjacent the binding site of the protein. In some cases, the analyte, when present, may bind to the paramagnetic metal ion. Proteins may be particularly useful since they can be readily modified to suit a particular application, for example, via various genetic engineering methods. For example, the binding specificity of a particular protein may be modified such that the protein binds an analyte of interest. Additionally, proteins may be delivered to a subject (e.g., human) with relatively low levels of toxicity. In some cases, the species is any protein, except hemoglobin or myoglobin, including mutants and variants thereof. In some cases, the protein is a genetically-engineered molecular protein, as described more fully below.

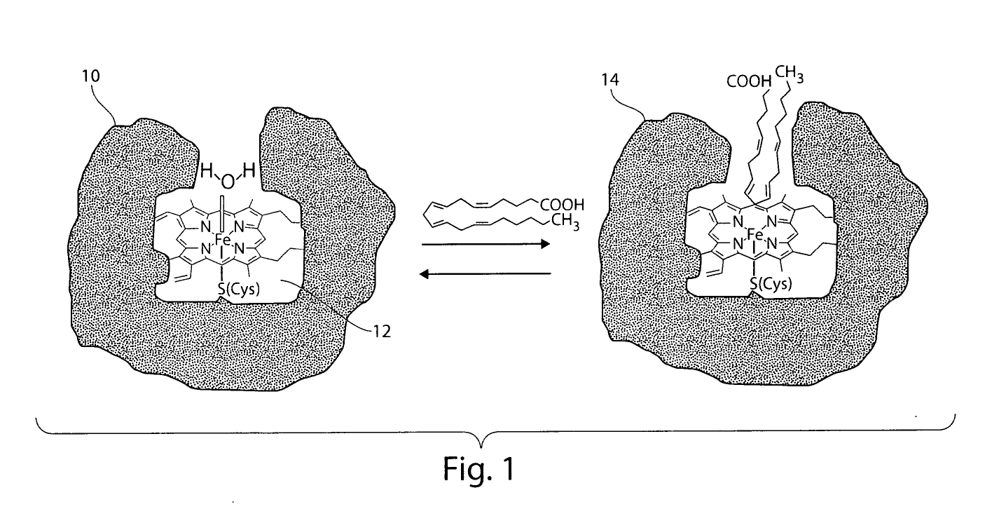

In an illustrative embodiment, FIG. 1 shows a protein-based sensor 10 comprising a binding site 12. In the absence of an analyte, a water molecule may be bound to binding site 12, i.e., via a paramagnetic metal ion, such that the sensor 10 generates a first MRI signal upon exposure to MRI conditions. In the presence of an analyte, the analyte may replace the water molecule and may bind the paramagnetic metal ion to form analyte-bound, protein-based sensor 14, which may generate a second MRI signal upon exposure to said MRI conditions. The second MRI signal may be shifted relative to the first MM signal, such that the shift indicates the presence and/or amount of analyte present in the sample. It should be understood that the shift in the MRI signal may be produced by any interaction of the protein-based sensor with an analyte, including replacement of a ligand (e.g., water) by the analyte, a change in the number of coordination sites of a metal ion in or adjacent the binding site upon interaction with the analyte, other chemical reactions that may occur within or adjacent the binding site as a result of the presence of the analyte, etc.

In some embodiments, the interaction between the analyte and the binding site of a protein may comprise formation of a bond, such as a covalent bond (e.g. carbon-carbon, carbon-oxygen, oxygen-silicon, sulfur-sulfur, phosphorus-nitrogen, carbon-nitrogen, metal-oxygen or other covalent bonds), an ionic bond, a hydrogen bond (e.g., between hydroxyl, amine, carboxyl, thiol and/or similar functional groups, for example), a dative bond (e.g. complexation or chelation between metal ions and monodentate or multidentate ligands), or the like. The interaction may also comprise Van der Waals interactions. In one embodiment, the interaction comprises coordination between a metal ion of the binding site and an analyte. The binding site may also interact with an analyte via a binding event between pairs of biological molecules. For example, the protein may comprise an entity, such as biotin that specifically binds to a complementary entity, such as avidin or streptavidin, on a target analyte.

In some cases, the binding site may comprise a biological or a chemical molecule able to bind to another biological or chemical molecule. For example, the binding site may comprise a functional group, such as a thiol, aldehyde, ester, carboxylic acid, hydroxyl, or the like, wherein the functional group forms a bond with the analyte. In some cases, the binding site may be an electron-rich or electron-poor moiety within the polymer, wherein interaction between the analyte and the conducting polymer comprises an electrostatic interaction.

The binding site may also be capable of biologically binding an analyte via an interaction that occurs between pairs of biological molecules including proteins, nucleic acids, glycoproteins, carbohydrates, hormones, and the like. Specific examples include an antibody/peptide pair, an antibody/antigen pair, an antibody fragment/antigen pair, an antibody/antigen fragment pair, an antibody fragment/antigen fragment pair, an antibody/hapten pair, an enzyme/substrate pair, an enzyme/inhibitor pair, an enzyme/cofactor pair, a protein/substrate pair, a nucleic acid/nucleic acid pair, a protein/nucleic acid pair, a peptide/peptide pair, a protein/protein pair, a small molecule/protein pair, a glutathione/GST pair, an anti-GFP/GFP fusion protein pair, a Myc/Max pair, a maltose/maltose binding protein pair, a carbohydrate/protein pair, a carbohydrate derivative/protein pair, a metal binding tag/metal/chelate, a peptide tag/metal ion-metal chelate pair, a peptide/NTA pair, a lectin/carbohydrate pair, a receptor/hormone pair, a receptor/effector pair, a complementary nucleic acid/nucleic acid pair, a ligand/cell surface receptor pair, a virus/ligand pair, a Protein A/antibody pair, a Protein G/antibody pair, a Protein L/antibody pair, an Fc receptor/antibody pair, a biotin/avidin pair, a biotin/streptavidin pair, a drug/target pair, a zinc finger/nucleic acid pair, a small molecule/peptide pair, a small molecule/protein pair, a small molecule/target pair, a carbohydrate/protein pair such as maltose/MBP (maltose binding protein), a small molecule/target pair, or a metal ion/chelating agent pair. It should be understood that any interaction which binds an analyte to the protein may be used, such that the binding of the analyte produces a change or shift in the MRI signal of the protein.

As used herein, the term “genetically-engineered protein” refers to proteins generated (e.g., synthesized) using genetic engineering methods including directed evolution, site-directed mutagenesis, yeast or phage display, and other screening-based methods known in the art. In some cases, the genetically-engineered protein is generated using directed evolution. Typically, the genetically-engineered protein is a mutant protein, i.e., a protein whose structure has been modified. The metalloproteins may be selected and/or engineered such that its MRI contrast properties are affected by the presence of a particular analyte. For example, prior to modification by genetic engineering, a protein may specifically bind a first analyte, such that one or more MRI properties may be affected in response to the binding of the first analyte. The protein may then be genetically engineered (e.g., evolved) to exhibit specificity for a second analyte, different from the first analyte. Proteins of the invention may advantageously be modified to have binding specificity for a wide range of analytes. In some embodiments, the protein may be introduced or exposed to a biological sample in the form of a nucleic acid molecule (e.g. gene), as described more fully below. Examples of proteins that may be genetically-engineered include, but are not limited to, a cytochrome, tyrosine hydroxylase, or phenylalanine hydroxylase. In a particular embodiment, the genetically-engineered protein is P450 BM3 or mutants thereof.

Methods of the invention may be useful in determining a broad range of analytes. In some cases, the analyte may be an organic analyte. In some embodiments, the analyte may be a neurotransmitter analyte. As used herein, the term “neurotransmitter analyte” refers to any species capable of relaying, modulating, and other otherwise facilitating transmittal of an electrical signal between a neuron and another species, such as a cell. A neurotransmitter analyte may be produced endogenously or exogenously. Examples of neurotransmitter analytes include, but are not limited to, ions, amino acids, peptides, purines, hormones, gastrins, fatty acids, amines, other small molecules, is sufficient for many purposes. Specific examples of neurotransmitter analytes include zinc ions, acetylcholine, norepinephrine (NE), epinephrine, dopamine (DA), serotonin (5-HT), melatonin, octopamine, tyramine, glutamic acid, gamma-aminobutyric acid (GABA), aspartic acid, glutamic acid. glycine, N-acetylaspartylglutamate, adenosine, cholecystokinin, vasopressin, somatostatin, neurotensin, oxytocin, neurophysin I, neurophysin II, corticotropin dynorphin, endorphin, enkephaline, secretin, motilin, glucagon, neurokinin A, neurokinin B histamine, ATP, GTP, LH, insulin, and derivatives thereof. It should be understood that other analytes may also be determined, such as nitric oxide, carbon monoxide, carbon doixide, oxygen, and the like.

Potential applications of methods of the invention include a wide variety of possible in vivo molecular imaging studies in animals (e.g. studies relating neural molecular activity to behavior, genes, etc), diagnostic neural imaging of patients with neurological disorders such as epilepsy and depression (e.g. to locate seizure foci for excision), implementation of non-invasive brain-computer interfaces (e.g. in patients with spinal cord injury, ALS or MS), and the like.

Protein-based MRI contrast agents and/or sensors, as described herein, may be synthesized from scratch or may be based on suitable natural proteins such as the enzymes cytochromes (e.g. cytochrome P450-BM3), tyrosine hydroxylases, phenylalanine hydroxylases, etc. Their specificity towards particular molecular targets may be tuned by rational design or directed evolution. Expression of metalloprotein MRI contrast agents may be targeted to specific tissues, cell types or compartments by established gene delivery methods (e.g. adenovirus), or the contrast agents can be expressed in bulk and delivered as intact proteins to sites of interest.

In some embodiments, directed evolution may be used to design and synthesize proteins for use as MRI contrast agents and/or sensors. For example, a mutant library may be generated by error prone PCR, saturation mutagenesis, homologous recombination, or the like, and may be transformed into E. coli. High-throughput screening of mutant libraries may be conducted, by selecting the mutants which exhibit enhanced affinity and selectivity for a particular analyte, such as dopamine or seratonin. The individual mutants may then be expressed in 96-well format, and subcultures of E. coli expressing the mutants may be lysed. A spectroscopic screen may then be used to identify mutants showing desired activity. For example, methods of the invention may comprise monitoring the absorbance spectrum of metalloproteins of the invention, wherein the absorbance spectrum is generated by the metalloprotein itself, without need for an additional or surrogate entity (e.g., chromophore) associated with the metalloprotein to generate an optical signal.

Proteins of the invention may be introduced into, or exposed to, the biological sample using various methods known in the art, as described more fully below. Some embodiments of the invention may involve direct introduction of the protein molecule into the biological sample, or, via introduction of the protein molecule into the biological sample via the introduction of a nucleic acid molecule which encodes the protein into the biological sample, either virally or non-virally mediated (e.g., gene delivery).

Some embodiments of the invention also provide polypeptides (e.g., proteins) as described herein and the nucleic acid molecules that encode them, as well as related nucleic acid and polypeptide sequences. The invention further relates to the use of the nucleic acid molecules, polypeptides and fragments thereof in methods and compositions for use as MRI sensors and/or diagnostic tools

In some embodiments, the invention provides polypeptides (e.g., proteins) as well as the nucleic acid molecules that encode the polypeptides. As used herein, the “nucleic acid molecules that encode” means the nucleic acid molecules that code for the polypeptides or fragments thereof. These nucleic acid molecules may be DNA or may be RNA (e.g., mRNA). The nucleic acid molecules of the invention also encompass variants of the nucleic acid molecules described herein. These variants may be splice variants or allelic variants of certain sequences provided. Variants of the nucleic acid molecules of the invention are intended to include homologs and alleles which are described further below.

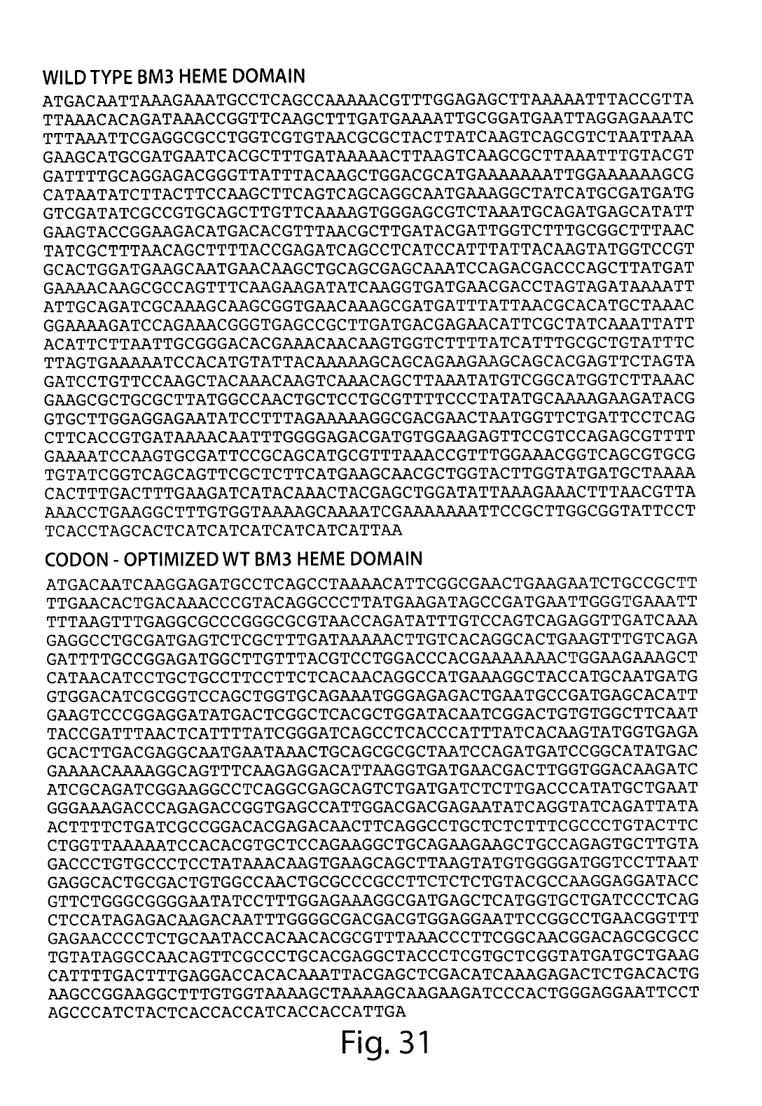

In some embodiments, the invention provides isolated nucleic acid molecules that encode the polypeptides described herein. The isolated nucleic acid molecules may include (a) nucleotide sequences set forth as any of the nucleotide sequences in FIG. 31; (b) isolated nucleic acid molecules which hybridize under highly stringent conditions to the nucleic acid molecules of (a) and, preferably, which code for a polypeptide; (c) nucleic acid molecules that differ from (a) or (b) due to the degeneracy of the genetic code; and (d) full-length complements of (a), (b) or (c).

As used herein the term “isolated nucleic acid molecule” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a small percentage of the material in the cell in which it resides. Such a nucleic acid is isolated, however, as the term is used herein because it is readily manipulable by standard techniques known to those of ordinary skill in the art.

The nucleic acid molecules of the invention also encompass homologs and alleles which can be identified by conventional techniques. Identification of human and other organisms' homologs of polypeptides will be familiar to those of skill in the art. In general, nucleic acid hybridization is a suitable method for identification of homologous sequences of another species (e.g., human, cow, sheep, dog, rat, mouse), which correspond to a known sequence. Standard nucleic acid hybridization procedures can be used to identify related nucleic acid sequences of selected percent identity. For example, one can construct a library of cDNAs reverse transcribed from the mRNA of a selected tissue and use the nucleic acid molecules identified herein to screen the library for related nucleotide sequences. The screening preferably is performed using high-stringency conditions to identify those sequences that are closely related by sequence identity. Nucleic acids so identified can be translated into polypeptides (e.g., proteins) and the polypeptides can be tested for their ability to bind a particular analyte of interest.

The term “high stringency” as used herein refers to parameters with which the art is familiar. Nucleic acid hybridization parameters may be found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, high-stringency conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH2PO4 (pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1×SDS at temperatures up to 68° C.

There are other conditions, reagents, and so forth that can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of the nucleic acids of the invention (e.g., by using lower stringency conditions). The skilled artisan also is familiar with the methodology for screening cells and libraries for expression of such molecules, which then are routinely isolated, followed by isolation of the pertinent nucleic acid molecule and sequencing. In general, homologs and alleles typically will share at least 90% nucleotide identity and/or amino acid identity to the sequences of nucleic acids and polypeptides, respectively, in some instances will share at least 95% nucleotide identity and/or amino acid identity, in other instances will share at least 97% nucleotide identity and/or amino acid identity, in other instances will share at least 98% nucleotide identity and/or amino acid identity, and in other instances will share at least 99% nucleotide identity and/or amino acid identity. The homology can be calculated using various, publicly available software tools developed by NCBI (Bethesda, Md.) that can be obtained through the internet. Exemplary tools include the BLAST system available from the website of the National Center for Biotechnology Information (NCBI) at the National Institutes of Health. Pairwise and ClustalW alignments (BLOSUM30 matrix setting) as well as Kyte-Doolittle hydropathic analysis can be obtained using a number of sequence analysis software programs, such as the MacVector sequence analysis software (Accelrys Software Inc., San Diego, Calif.). Watson-Crick complements of the foregoing nucleic acids also are embraced by the invention.

In some embodiments, unique fragments are provided which include unique fragments of the nucleotide sequences of the invention and complements thereof. A unique fragment is one that is a ‘signature’ for the larger nucleic acid. It, for example, is long enough to assure that its precise sequence is not found in molecules outside of the nucleic acid molecules that encode the polypeptides defined above. Those of ordinary skill in the art may apply no more than routine procedures to determine if a fragment is unique within the human genome. In some instances the unique fragment is at least about 5, 6, 7, 8, 9, 10, 11, 12, 13, 14, 15, 16, 17, 18, 19, 20, 25, 30, 35, 40, 45, 50, 75, or 100 amino acids in length.

Unique fragments can be used as probes in Southern blot assays to identify such nucleic acid molecules, or can be used as probes in amplification assays such as those employing the polymerase chain reaction (PCR), including, but not limited to RT-PCR and RT-real-time PCR. As known to those skilled in the art, large probes such as 200 nucleotides or more are preferred for certain uses such as Southern blots, while smaller fragments will be preferred for uses such as PCR. Unique fragments also can be used to produce fusion proteins for generating antibodies or determining binding of the polypeptide fragments, or for generating immunoassay components. In screening for genes, a Southern blot may be performed using the foregoing conditions, together with a detectably labeled probe (e.g., radioactive or chemiluminescent probes). After washing the membrane to which the DNA is finally transferred, the membrane can be placed against X-ray film or analyzed using a phosphorimager device to detect the radioactive or chemiluminescent signal. In screening for the expression of nucleic acids, Northern blot hybridizations using the foregoing conditions can be performed on samples taken from cancer patients or subjects suspected of having a condition characterized by abnormal cell proliferation or neoplasia. Amplification protocols such as polymerase chain reaction using primers that hybridize to the sequences presented also can be used for detection of the genes or expression thereof.

Identification of related sequences can also be achieved using polymerase chain reaction (PCR) and other amplification techniques suitable for cloning related nucleic acid sequences. Preferably, PCR primers are selected to amplify portions of a nucleic acid sequence believed to be conserved (e.g., a catalytic domain, a DNA-binding domain, etc.). Again, nucleic acids are preferably amplified from a tissue-specific library (e.g., testis). One also can use expression cloning utilizing the antisera described herein to identify nucleic acids that encode related antigenic proteins in humans or other species using the SEREX procedure to screen the appropriate expression libraries. (See: Sahin et al., 1995, Proc. Natl. Acad. Sci. U.S.A. 92:11810-11813).

The invention also includes degenerate nucleic acids that include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating polypeptide. Similarly, nucleotide sequence triplets which encode other amino acid residues include, but are not limited to: CCA, CCC, CCG, and CCT (proline codons); CGA, CGC, CGG, CGT, AGA, and AGG (arginine codons); ACA, ACC, ACG, and ACT (threonine codons); AAC and AAT (asparagine codons); and ATA, ATC, and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides modified nucleic acid molecules, which include additions, substitutions and deletions of one or more nucleotides (preferably 1-20 nucleotides). In preferred embodiments, these modified nucleic acid molecules and/or the polypeptides they encode retain at least one activity or function of the unmodified nucleic acid molecule and/or the polypeptides, such as antigenicity, receptor binding, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules that encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules that encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on.

In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of activity or structural relation to the nucleic acids and/or polypeptides disclosed herein. As used herein the terms: “deletion”, “addition”, and “substitution” mean deletion, addition, and substitution changes to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more nucleic acids of a sequence of the invention.

In some embodiments, an expression vector comprising any of the isolated nucleic acid molecules of the invention, preferably operably linked to a promoter is provided. In a related aspect, host cells transformed or transfected with such expression vectors also are provided. As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids, and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art, e.g., beta-galactosidase or alkaline phosphatase, and genes which visibly affect the phenotype of transformed or transfected cells, hosts, colonies or plaques, e.g., green fluorescent protein. Preferred vectors are those capable of autonomous replication and expression of the structural gene products present in the DNA segments to which they are operably joined.

As used herein, a coding sequence and regulatory sequences are said to be “operably joined” when they are covalently linked in such a way as to place the expression or transcription of the coding sequence under the influence or control of the regulatory sequences. As used herein, “operably joined” and “operably linked” are used interchangeably and should be construed to have the same meaning. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region is capable of effecting transcription of that DNA sequence such that the resulting transcript can be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Often, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

It will also be recognized that the invention embraces the use of the nucleic acid molecules and genomic sequences described herein in expression vectors, as well as to transfect host cells and cell lines, be these prokaryotic, e.g., E. coli, or eukaryotic, e.g., CHO cells, COS cells, yeast expression systems, and recombinant baculovirus expression in insect cells. Especially useful are mammalian cells such as human, mouse, hamster, pig, goat, primate, etc. They may be of a wide variety of tissue types, including mast cells, fibroblasts, oocytes, and lymphocytes, and may be primary cells and cell lines. Specific examples include dendritic cells, peripheral blood leukocytes, bone marrow stem cells and embryonic stem cells. The expression vectors require that the pertinent sequence, i.e., those nucleic acids described supra, be operably linked to a promoter.

Expression vectors containing all the necessary elements for expression are commercially available and known to those skilled in the art. Cells are genetically engineered by the introduction into the cells of heterologous DNA or RNA encoding a polypeptide, a mutant polypeptide, fragments, or variants thereof. The heterologous DNA or RNA is placed under operable control of transcriptional elements to permit the expression of the heterologous DNA in the host cell.

Preferred systems for mRNA expression in mammalian cells are those such as pcDNA3.1 and pCDM8 (Invitrogen) that contain a selectable marker (which facilitates the selection of stably transfected cell lines) and contain the human cytomegalovirus (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1, which stimulates efficiently transcription in vitro. The plasmid is described by Mizushima and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant is described by Warnier et al., in intradermal injection in mice for immunization against P1A (Int. J. Cancer, 67:303-310, 1996).

The invention also embraces kits termed expression kits, which allow the artisan to prepare a desired expression vector or vectors. Such expression kits include at least separate portions of each of the previously discussed coding sequences. Other components may be added, as desired, as long as the previously mentioned sequences, which are required, are included.

The invention also includes kits for amplification of a nucleic acid, including at least one pair of amplification primers which hybridize to a nucleic acid. The primers preferably are about 12, 13, 14, 15, 16, 17, 18, 19, 20, 21, 22, 23, 24, 25, 26, 27, 28, 29, 30, 31 or 32 nucleotides in length and are non-overlapping to prevent formation of “primer-dimers”. One of the primers will hybridize to one strand of the nucleic acid and the second primer will hybridize to the complementary strand of the nucleic acid, in an arrangement that permits amplification of the nucleic acid. Selection of appropriate primer pairs is standard in the art. For example, the selection can be made with assistance of a computer program designed for such a purpose, optionally followed by testing the primers for amplification specificity and efficiency.

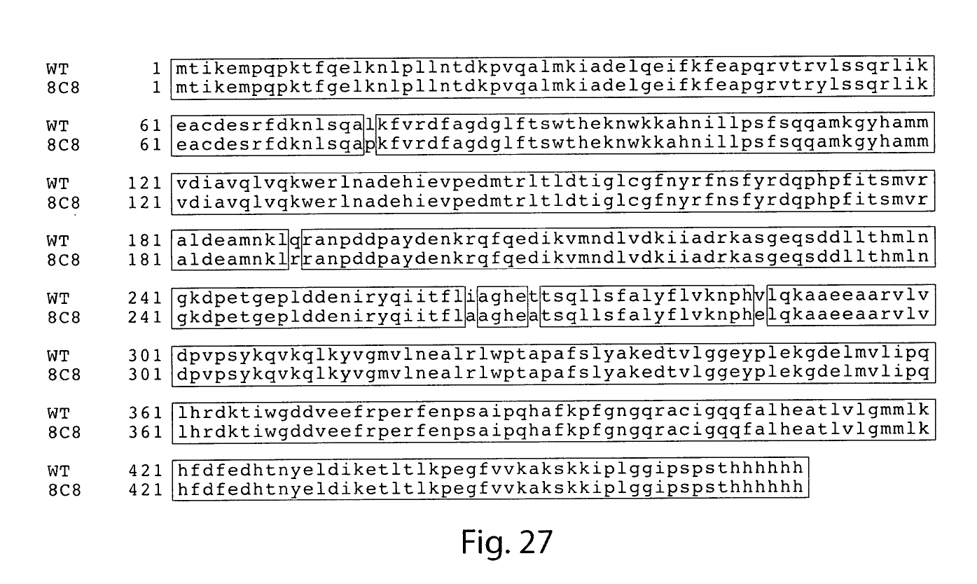

The invention, in another aspect provides isolated polypeptides (including whole proteins and partial proteins) encoded by the foregoing nucleic acids. Examples of the amino acid sequences encoded by the foregoing nucleic acids are set forth as any of the amino acid sequence in FIG. 31. The amino acids of the invention are also intended to encompass amino acid sequences that result from the translation of the nucleic acid sequences provided herein in a different reading frame. In one embodiment, a polypeptide is provided which comprises the polypeptide sequence set forth in FIG. 27.

The invention embraces variants of the polypeptides described above. As used herein, a “variant” of a polypeptide is a polypeptide which contains one or more modifications to the primary amino acid sequence of a polypeptide. Modifications which create a polypeptide variant can be made to a polypeptide 1) to reduce or eliminate an activity of a polypeptide; 2) to enhance a property of a polypeptide, such as protein stability in an expression system or the stability of protein-protein binding; 3) to provide a novel, or improved, activity or property to a polypeptide, such as binding site specificity for a desired analyte; or 4) other modifications which enhance one or more properties of the polypeptide.

In some cases, modifications to a polypeptide are made to the nucleic acid which encodes the polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the polypeptide amino acid sequence. One of skill in the art will be familiar with methods for predicting the effect on protein conformation of a change in protein sequence, and can thus “design” a variant polypeptide according to known methods. One example of such a method is described by Dahiyat and Mayo in Science 278:82-87, 1997, whereby proteins can be designed de novo. The method can be applied to a known protein to vary a only a portion of the polypeptide sequence.

Mutations of a nucleic acid which encode a polypeptide preferably preserve the amino acid reading frame of the coding sequence, and preferably do not create regions in the nucleic acid which are likely to hybridize to form secondary structures, such a hairpins or loops, which can be deleterious to expression of the variant polypeptide.

Mutations can be made by selecting an amino acid substitution, or by random mutagenesis of a selected site in a nucleic acid which encodes the polypeptide. Variant polypeptides are then expressed and tested for one or more activities to determine which mutation provides a variant polypeptide with the desired properties. Further mutations can be made to variants (or to non-variant polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a gene or cDNA clone to enhance expression of the polypeptide. The activity of variants of polypeptides can be tested by cloning the gene encoding the variant polypeptide into a bacterial or mammalian expression vector, introducing the vector into an appropriate host cell, expressing the variant polypeptide, and testing for a functional capability of the polypeptides as disclosed herein. Preparation of other variant polypeptides may favor testing of other activities, as will be known to one of ordinary skill in the art.

Those of ordinary skill in the art would also realize that conservative amino acid substitutions may be made in immunogenic polypeptides to provide functionally equivalent variants, or homologs of the foregoing polypeptides, i.e., the variants retain the functional capabilities of the immunogenic polypeptides. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the polypeptides disclosed herein and retain the specific antibody-binding characteristics of the antigens.

Conservative amino-acid substitutions in the amino acid sequence of polypeptides to produce functionally equivalent variants of polypeptides typically are made by alteration of a nucleic acid encoding a polypeptide. Such substitutions can be made by a variety of methods known to one of ordinary skill in the art. For example, amino acid substitutions may be made by PCR-directed mutation, site-directed mutagenesis according to the method of Kunkel (Kunkel, 1985, Proc. Nat. Acad. Sci. U.S.A. 82: 488-492), or by chemical synthesis of a gene encoding a polypeptide. Where amino acid substitutions are made to a small unique fragment of a polypeptide, such as an antigenic epitope recognized by autologous or allogeneic sera or T lymphocytes, the substitutions can be made by directly synthesizing the peptide. The activity of functionally equivalent variants of polypeptides can be tested by cloning the gene encoding the altered polypeptide into a bacterial or mammalian expression vector, introducing the vector into an appropriate host cell, expressing the altered polypeptide, and testing for a functional capability of the polypeptides as disclosed herein. Peptides that are chemically synthesized can be tested directly for function.

As used herein, a “subject” is preferably a human, non-human primate, cow, horse, pig, sheep, goat, dog, cat or rodent. In all embodiments, human subjects are preferred.

As used herein, a “biological sample” includes, but is not limited to: tissue, cells, and/or body fluid (e.g., serum, blood, lymph node fluid, etc.). The fluid sample may include cells and/or fluid. The tissue and cells may be obtained from a subject or may be grown in culture (e.g., from a cell line). As used herein, a biological sample is body fluid, tissue or cells obtained from a subject using methods well-known to those of ordinary skill in the related medical arts. Typically, a biological sample may be obtained by collecting a blood sample or a biopsy sample from a subject. The biological sample can include tumor tissue or cells, normal tissue or cells, or combinations thereof.

Various techniques may be employed for introducing nucleic acids of the invention into cells, depending on whether the nucleic acids are introduced in vitro or in vivo in a host. Such techniques include transfection of nucleic acid-CaPO4 precipitates, transfection of nucleic acids associated with DEAE, transfection or infection with the foregoing viruses including the nucleic acid of interest, liposome mediated transfection, and the like. For certain uses, it may be preferred to target the nucleic acid to particular cells. In such instances, a vehicle used for delivering a nucleic acid of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. Where liposomes are employed to deliver the nucleic acids of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

Compositions containing the nucleic acid molecules and polypeptides (e.g., proteins) of the invention are provided. The compositions contain any of the foregoing agents in a carrier, optionally a pharmaceutically acceptable carrier. When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term “carrier” denotes an organic or inorganic ingredient, natural or synthetic, with which the active ingredient is combined to facilitate the application. The components of the compositions also are capable of being co-mingled with the molecules of the present invention, and with each other, in a manner such that there is no interaction which would substantially impair the ability of compounds of the invention to interact with an analyte, as described herein.

Compositions of the invention can be administered by any conventional route, including injection or by gradual infusion over time. The administration may, for example, be oral, intravenous, intratumoral, intraperitoneal, intramuscular, intracavity, subcutaneous, or transdermal. The composition can be administered in an effective amounts. An “effective amount” is that amount of a polypeptide composition that alone, or together with further doses, produces the desired MRI signal, for example, for an MRI contrast image and/or for determination of an analyte.

The compositions may contain suitable buffering agents, including: acetic acid in a salt; citric acid in a salt; boric acid in a salt; and phosphoric acid in a salt. The compositions also may contain, optionally, suitable preservatives, such as: benzalkonium chloride; chlorobutanol; parabens and thimerosal. The compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the protein-based sensor. Other compositions include suspensions in aqueous liquids or non-aqueous liquids such as a syrup, elixir or an emulsion.

Compositions for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, and lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases, and the like.

The compositions of the invention may be administered alone, in combination with each other, and/or in combination with other drug therapies and/or treatments. The invention also provides a kit comprising one or more containers comprising one or more of the compounds or agents of the invention. Additional materials may be included in any or all kits of the invention, and such materials may include, but are not limited to buffers, water, enzymes, tubes, control molecules, etc. The kit may also include instructions for the use of the one or more compounds or agents of the invention for determination of an analyte.

The foregoing kits can include instructions or other printed material on how to use the various components of the kits for diagnostic purposes.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### EXAMPLES

### Example 1

The following example describes the synthesis of a metalloprotein based on cytochrome P450-BM3, and its use as a prototypical metalloprotein MRI contrast agent and sensor with tunable sensitivity. BM3 is a water-soluble fatty acid monooxygenase from Bacillus megaterium consisting of a heme domain and a reductase domain. The heme domain of BM3 contains a paramagnetic iron(III) atom that, in the enzyme's substrate-free state, is coordinated by a water molecule that becomes displaced by substrate binding, as shown in FIG. 1. The substrate-dependent switching of the interaction between water molecules and the paramagnetic Fe³⁺ can enable the BM3 heme domain to act as a T1 contrast agent sensitive to substrates. However, to make this sensor useful for other analytes of interest, such as the neurotransmitters dopamine (DA) and serotonin (5-HT), the binding specificity of the BM3 heme domain was altered to bind analytes other than long-chain fatty acid substrates.

Previous studies have employed directed evolution to alter the binding specificity of BM3 to create catalysts for hydroxylation of various non-native substrates, ranging from short alkanes to larger aromatic drugs. In directed evolution, random mutations are introduced into a protein (e.g., BM3) and the resulting mutants are screened in high throughput for activity on a desired novel substrate. The most active mutants are then selected for further mutagenesis and screening in an iterative process, until the desired level of activity is attained.

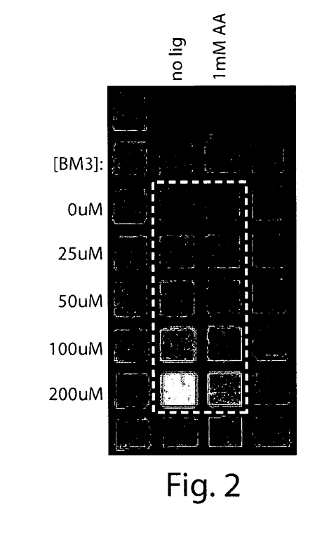

Using a similar approach, BM3 was evolved to bind neurotransmitters such as DA, 5-HT, or other molecules of interest. To test the idea that cytochrome P450-BM3 could produce MRI contrast in a ligand binding-dependent manner, MRI imaging of the wild type BM3 was performed in 384-well microtiter plates in a small-bore 4.7 T magnet (wild type BM3 provided by Frances Arnold's laboratory at Caltech). To determine the effects of ligand binding on relaxivity, a subset of samples was mixed with 1 mM arachidonic acid (AA), a native ligand known to bind this enzyme with high affinity. Since BM3 has a water molecule coordinated to the heme iron in its ligand-free state that gets displaced during ligand binding, mixing BM3 with ligand was expected to cause a decrease in T1 relaxivity, depending on the ability of water molecules to coordinate to the paramagnetic species. FIG. 2 displays a microplate MRI image acquired using a T1-weighted acquisition sequence, showing a decrease in signal when the BM3 is bound by AA. The use of a concentration series in both ligand-free and ligand-bound conditions allowed an accurate estimation of T1 relaxivity, which was 1.66 mM⁻¹ s⁻¹ and 2.64 mM⁻¹ s⁻¹ with and without the ligand, respectively. These values were within the range of functional synthetic sensors that have previously been applied in vivo, suggesting that BM3 can serve as a viable basis for a molecular sensor using MRI. For example, a beta-galactosidase-activated sensor developed by the Meade group, showed relaxivities of 0.903 mM⁻¹ s⁻¹ and 2.72 mM⁻¹ s⁻¹ before and after activation.

### Example 2

The following example describes the study of natural and novel ligand binding to the BM3 heme domain, as well as concentration-dependent changes in MRI contrast and optical absorption.

To determine the extent of that the interaction between exchanging water molecules at the axial site and the heme iron atom promotes T₁ relaxation in aqueous solutions, a spin echo MRI pulse sequence was used to measure the proton relaxation rate as a function of protein concentration in phosphate buffered saline solution (PBS); the slope of this relationship (T₁ relaxivity, or r₁) provided a standard measure of the strength of a contrast agent.

Samples (60-100 microliters) were arrayed into microtiter plates and placed in a 40-cm-bore Bruker (Billerica, Mass.) Avance 4.7 T MRI scanner, equipped with a 10 cm inner diameter birdcage resonator radiofrequency coil and 26 G/cm triple axis gradients. Unused wells of the microtiter plates were filled with phosphate buffered saline, and imaging was performed on a 2-mm slice through the sample. A T₁-weighted spin echo pulse sequence was used; echo time (TE) was 10 ms, and repetition times (TR) ranged from 77 ms to 5 s. Data matrices of 512×128 points were acquired and zero-filled to 1024×512 points, where the second dimension corresponds to the phase encoding direction. Images were reconstructed and analyzed using custom routines running in Matlab. Relaxation rates and relaxivities reported here were calculated by exponential fitting to the image data. Contrast was adjusted to produce MRI images presented in the figures.

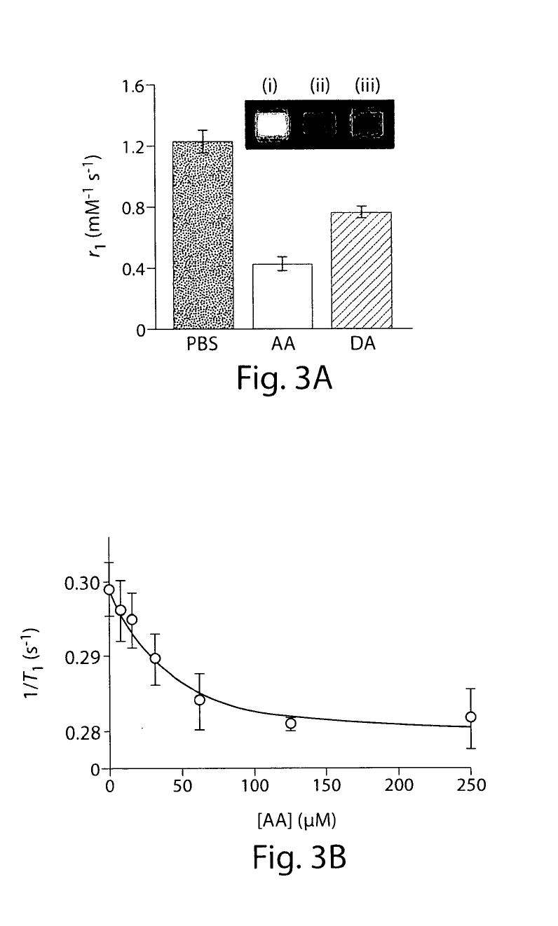

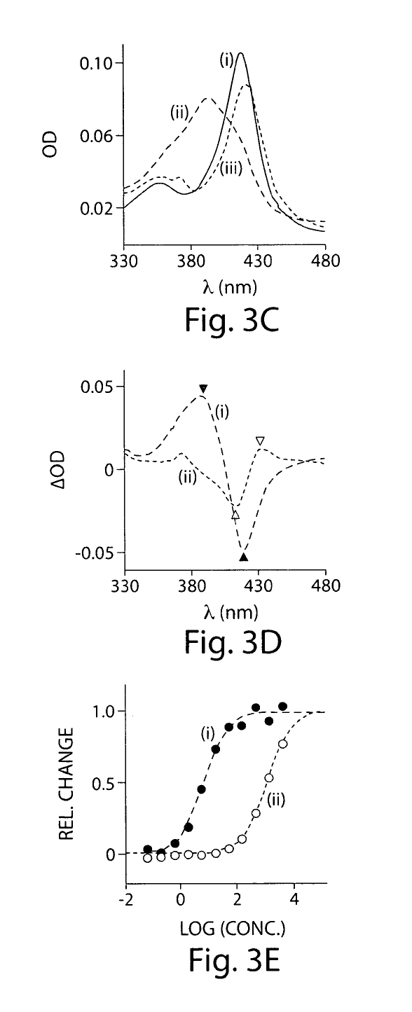

DA and related compounds were tested for the ability to serve as unnatural ligands for BM3h. As measured by MRI, addition of 1 mM DA to BM3h in fact induced a drop in r₁, to 0.76±0.03 mM⁻¹ s⁻¹. Binding of AA is known to induce a change (blue shift) in BM3h's optical absorbance spectrum, because of perturbation of the electronic environment of the heme chromophore. FIG. 3A shows a graph of the T₁ relaxivity (r₁) of BM3h in PBS solution, and in the presence of 400 micromolar AA or 1 mM DA, with the inset showing a T₁-weighted spin echo MRI image intensity (TE/TR=10/477 ms) of microtiter plate wells containing 0.24 mM BM3h in PBS alone (i), or in the presence of 0.4 mM AA (ii) or 1 mM DA (iii). FIG. 3B shows a plot of T₁ relaxation rates (1/T₁) measured from solutions of 28.5 micromolar BM3h incubated with solutions of AA at various concentrations (e.g., 0-250 micromolar AA). For BM3h in the absence of ligands, an r₁ value of 1.23±0.07 mM⁻¹ s⁻¹ was obtained. Addition of a saturating quantity of natural BM3 substrate, arachidonic acid (AA, 400 uM concentration), resulted in an r₁ of 0.42±0.05 mM⁻¹ s⁻¹. (FIG. 3A) This ligand-dependent change in relaxivity enabled quantitative sensing of AA using MRI, and suggested that BM3h could serve as a platform for molecular sensor engineering.

To determine whether low affinity binding by DA also influences optical absorption by BM3h, spectra were obtained in the presence and absence of 1 mM DA. FIG. 3C shows optical absorbance spectra of 1 micromolar BM3h measured alone (i), and following addition of 0.40 mM AA (ii) or 1 mM DA (iii). The interaction produced a significant red shift of λₘₐₓ, from 419 to 422 nm. (FIG. 3C) Red shifting of iron porphyrin absorption spectra has been interpreted as indicating direct coordination to the heme iron and suggested in this case that 1 mM DA directly replaces water as an axial metal ligand in BM3h. This model is consistent with the established propensity of catechols to bind to iron and other transition metals. These considerations strongly suggested that MRI and optical readouts of ligand binding to BM3h both report interactions with the heme site and that binding measurements obtained with either modality are comparable indicators of these interactions.

The absorption difference at two wavelengths was monitored as a function of ligand concentration to determine binding isotherms for AA and DA. FIG. 3D shows difference spectra showing the change in BM3h absorbance as a function of wavelength upon addition of 0.40 mM AA (i) or 1 mM DA (ii). FIG. 3E shows normalized titration curves of BM3h binding to AA (i) or DA (ii). The optical signals used for titration analysis were formed by subtracting the minimum from the maximum of difference spectra, indicated as arrowheads in FIG. 3D, under each set of conditions. Error bars were smaller than the symbols. For BM3h, the apparent K_{d} for AA was 6.8±0.5 micromolar; for DA the K_{d} was 990±110 micromolar.

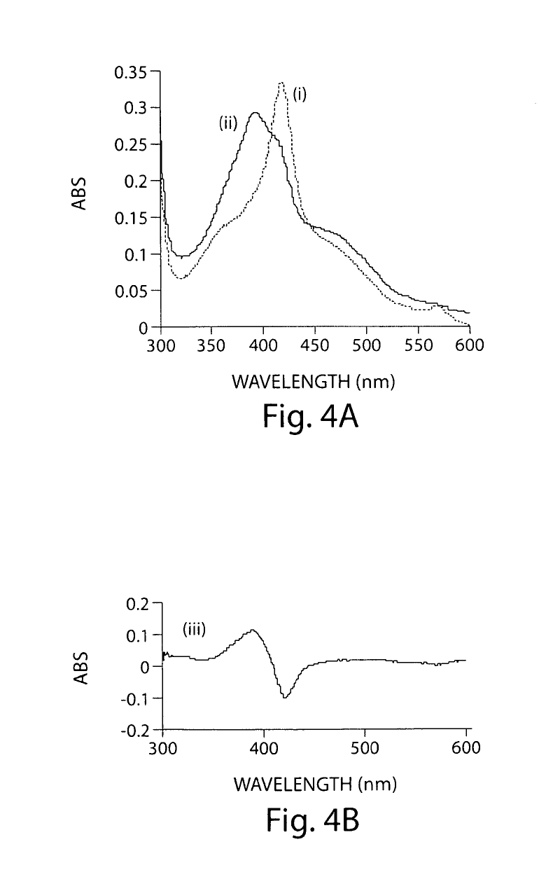

FIG. 4A shows the absorbance spectra of the evolved BM3 protein (e.g., BM3 mutant) upon exposure to (i) 0 mM arachidonic acid and (ii) 1 mM arachidonic acid, and FIG. 4B shows the corresponding absorbance difference spectrum.

Spectroscopy was then used to assay the binding of additional analytes to BM3 mutants. Several analytes were tested, including DA, 5-HT, and analytes which showed a structural resemblance to DA and 5-HT. Mutant BM3 clones were expressed in BL21 E. coli and purified on a nickel affinity column (via c-terminal 6H is tags). Concentrations were determined using a CO binding assay.

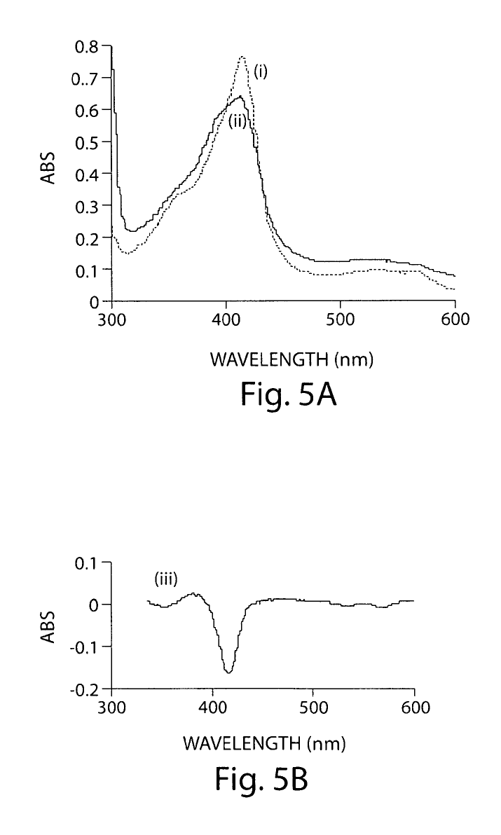

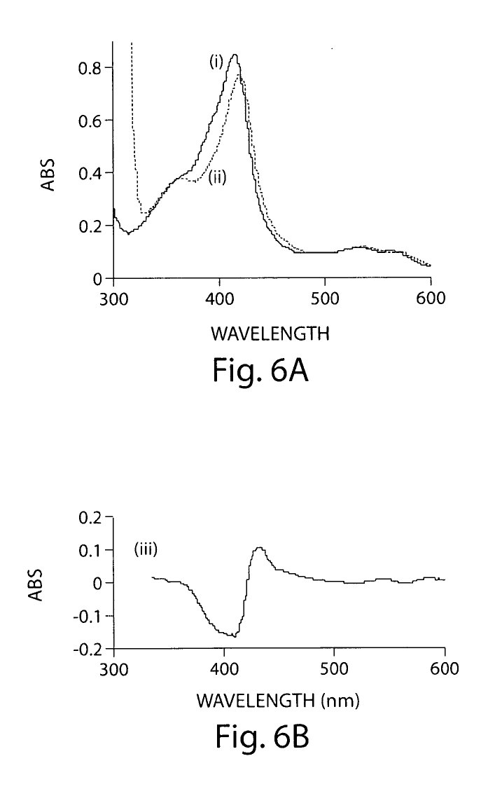

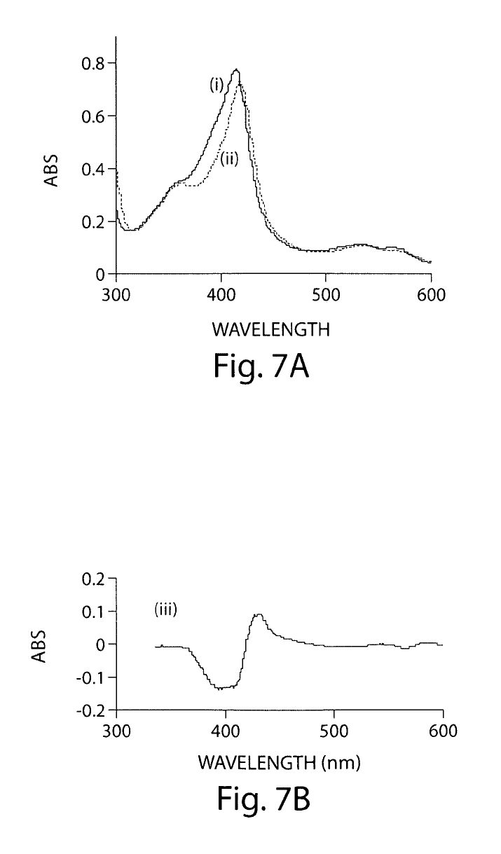

FIG. 5A shows the absorbance spectra of the mutant BM3 protein (C2B12H) upon exposure to (i) 0 mM chlorzoxazone and (ii) 2 mM chlorzoxazone, and FIG. 5B shows the corresponding absorbance difference spectrum. Binding of chlorzoxazone produced a characteristic spectral shift, with the difference spectrum showing a decrease at ˜420 nm and an increase at ˜390 nm. FIG. 6A shows the absorbance spectra of the mutant BM3 protein upon exposure to (i) 0 mM seratonin and (ii) 2 mM seratonin, and FIG. 6B shows the corresponding absorbance difference spectrum. FIG. 7A shows the absorbance spectra of the mutant BM3 protein upon exposure to (i) 0 mM dopamine and (ii) 2 mM dopamine, and FIG. 7B shows the corresponding absorbance difference spectrum. Dopamine and seratonin induced optical transitions, however, in contrast to the transitions produced by arachidonic acid and chlorzoxazone, dopamine and seratonin neurotransmitters caused the absorbance peak to shift into longer wavelengths. This effect was likely due to direct ligand coordination to the heme iron, a common mechanism for cytochrome inhibitors, as both dopamine and serotonin are known to be weak iron chelators. From the point of view of creating DA and 5-HT-specific GEMS, this mode of interaction may be beneficial. First, direct coordination of these ligands to the heme iron can ensure substantially complete blockage of water coordination, strengthening the T1 contrast mechanism. Second, this binding can provide an affinity platform upon which directed evolution can build to enhance affinity and selectivity for these and other neurotransmitter analytes. The experiments described above indicate that some advantageous characteristics of BM3h-based MRI sensors may include decreased affinity for AA, increased DA affinity, and enhanced relaxivity changes upon ligand binding.

### Example 3

The following example describes the study of mutant sensor proteins showing preferential MRI response to neurotransmitters and neurotransmitter analogs.

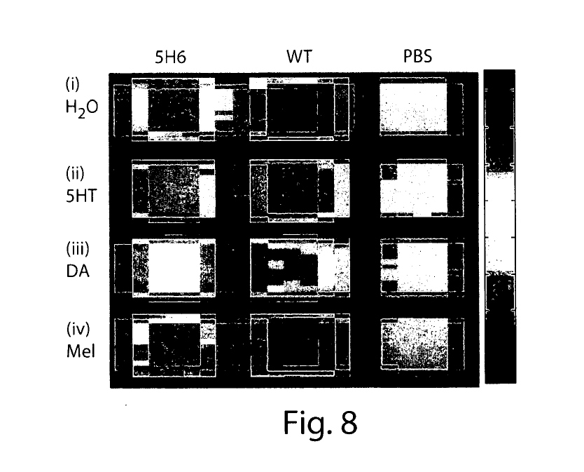

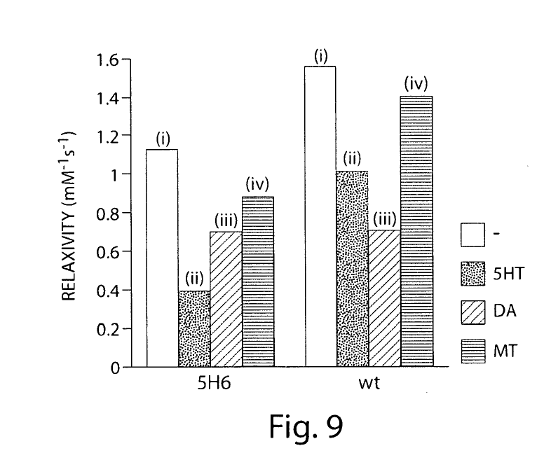

To confirm that the observed binding of BM3 by DA and 5-HT leads to the expected changes in MRI contrast, T1-weighted images of BM3 were acquired in the presence and absence of these molecules. FIG. 8 shows the T1-weighted images, while FIG. 9 shows the T1 relaxivities, for the wild type BM3 (WT) and mutant BM3 (5H6) proteins in the presence of (i) water, (ii) seratonin (5HT), (iii) dopamine (DA), and (iv) melatonin (Mel or MT). Both DA and 5-HT produced a significant decrease in T1 relaxivity. The relative sensitivity for DA versus 5-HT differed among BM3 variants. Of the two examples shown in FIGS. 8-9, the wild type BM3 protein appeared to have a greater response to DA, while the mutant BM3 protein (5H6) appeared to be more sensitive to 5-HT. Both showed only a minimal response to melatonin.

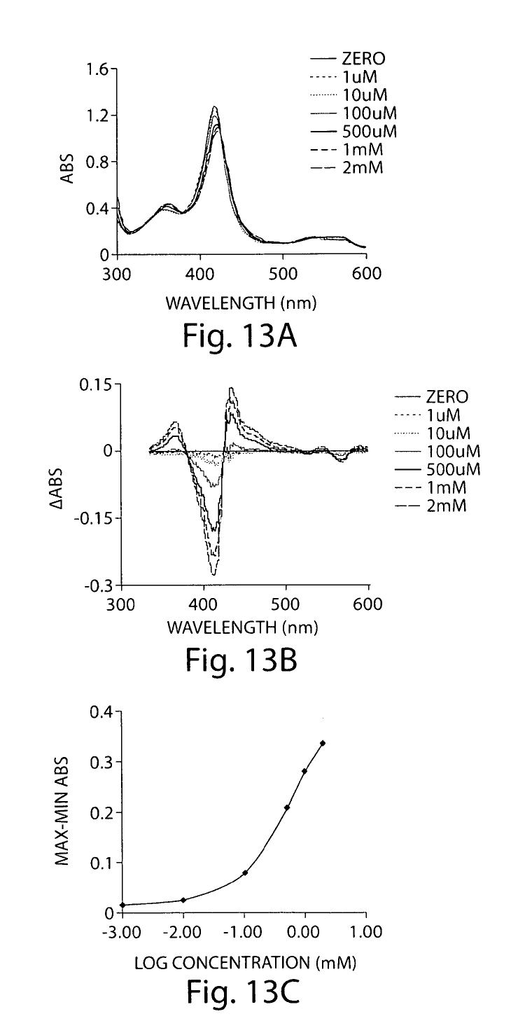

In another example, a spectroscopic assay based on analyte-induced absorbance shifts was employed to estimate binding affinities and ligand selectivity of the mutant proteins in the above Examples. FIG. 13A shows the absorption spectra of a wild type BM3 protein with increasing amounts of dopamine as the analyte. FIG. 13B shows the calculated change in absorbance upon each addition of analyte, and FIG. 13C shows the plot of the distance between the maximum and minimum points on each difference spectrum as a function of analyte concentration, generating a binding isotherm. This method allowed both an estimation of an analyte's dissociation constant and a determination of selectivity. In general, dopamine and serotonin showed binding affinities of around 1 mM.

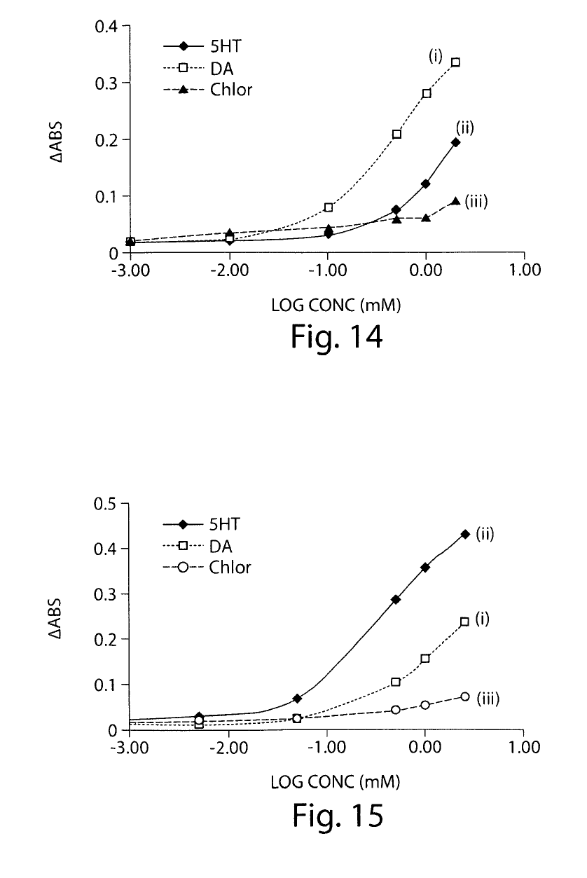

The analyte selectivity differences observed in the above Examples for the wild type and mutant BM3 proteins were further confirmed by this spectral method. FIG. 14 show the spectroscopic titration curve for a wild type BM3 protein in the presence of (i) dopamine, (ii) seratonin, and (iii) chlorzoxazone. FIG. 15 shows the spectroscopic titration curve for a mutant BM3 protein (5H6) in the presence of (i) dopamine, (ii) seratonin, and (iii) chlorzoxazone.

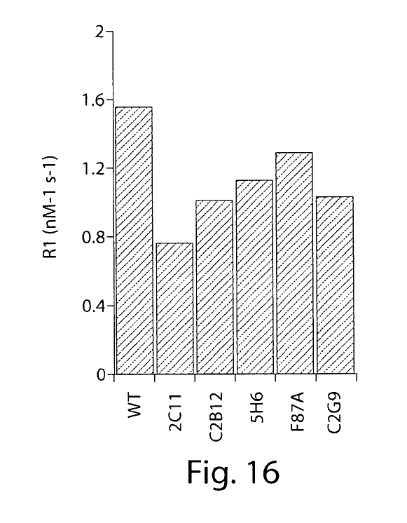

In addition to differences among mutants of BM3 in ligand binding affinity, differences in baseline (analyte-free) T1 relaxivity were observed for a series of mutant BM3 proteins, ranging from 0.76 to 1.56 mM⁻¹ s⁻¹, as shown by the graph in FIG. 16. The difference may be related to the rate of exchange of water molecules between the heme pocket of BM3 and bulk solvent.

In summary, P450-BM3 has been shown in the above Examples to be useful as an evolvable genetically encoded molecular sensor. BM3 mutants can produce significant analyte binding-dependent T1 contrast in MRI, and analyte binding can be assayed quantitatively using the method of spectroscopic titration. Additionally, non-native ligands, including analytes such as DA and 5-HT, produce changes in both spectroscopic and MRI responses of BM3. The mutants of BM3 display differences in selectivity for DA versus 5-HT and in their baseline T1 relaxivities.

### Example 4

The following example describes the study of selected sensor proteins showing strong, preferential MRI response to DA. In this example, two variants of the protein P450-BM3 were selected by a high throughput screening approach. The variants are referred to as BM3h-B7 and BM3h-8C8.

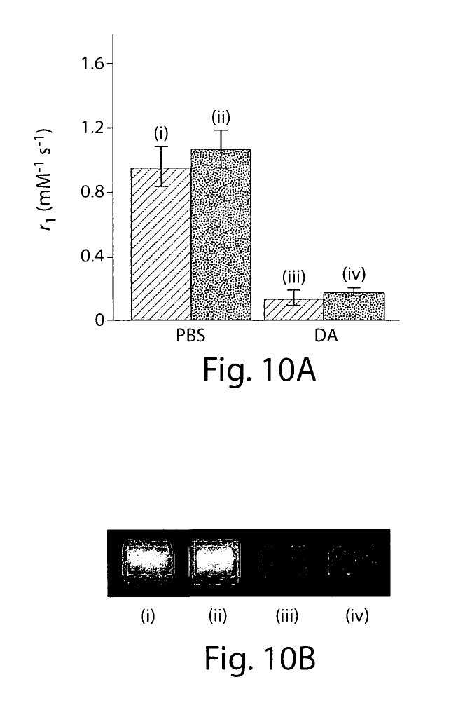

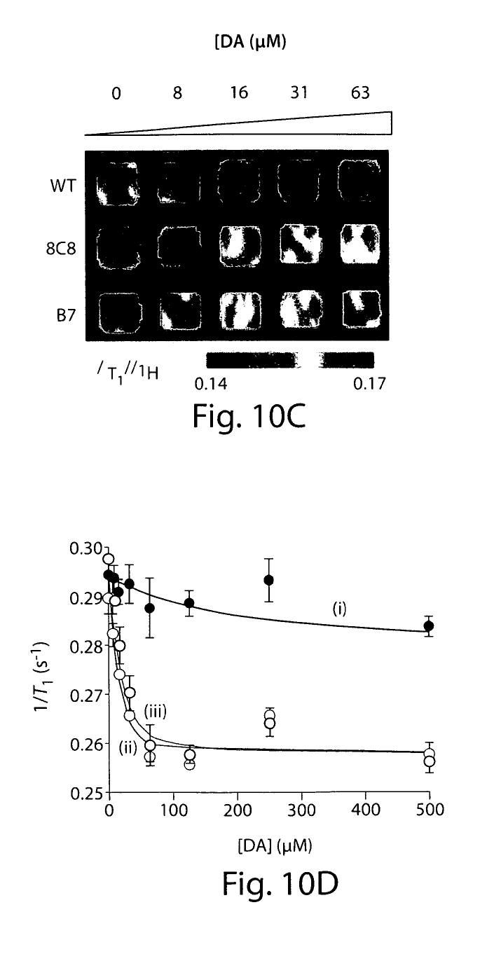

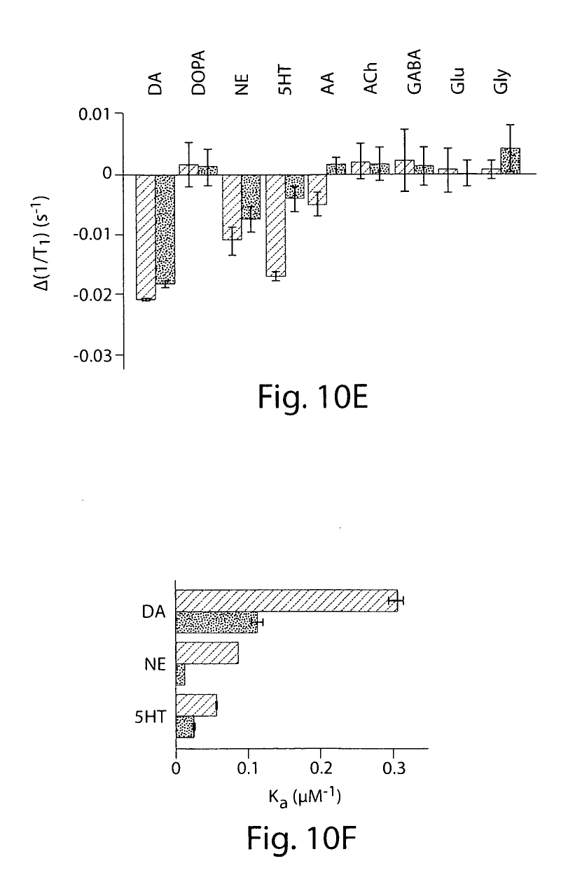

FIG. 10A shows a graph of the relaxivity values measured from (i) BM3h-B7 incubated in PBS alone, (ii) BM3h-B7 incubated in the presence of 400 micromolar DA, (iii) BM3h-8C8 incubated in PBS alone, and (iv) BM3h-8C8 incubated in the presence of 400 micromolar DA. FIG. 10B shows T₁-weighted MRI signals (TE/TR=10/477 ms) obtained from 195 micromolar samples of (i) BM3h-B7 incubated in PBS alone, (ii) BM3h-B7 incubated in the presence of 400 micromolar DA, (iii) BM3h-8C8 incubated in PBS alone, and (iv) BM3h-8C8 incubated in the presence of 400 micromolar DA. The mutant proteins selected after the fourth and fifth round of evolution in Example 9, denoted BM3h-8C8 and BM3h-B7, had dissociation constants of 8.9±0.7 micromolar and 3.3±0.1 micromolar, respectively, for DA, and 750±140 micromolar and 660±80 micromolar for AA. The T₁ relaxivity of BM3h-8C8 was 1.1±0.1 mM⁻¹ s⁻¹ in the absence of ligand, and 0.17±0.03 mM⁻¹ s⁻¹ in the presence of 400 micromolar DA. For BM3h-B7, the corresponding r₁ values were 0.96±0.13 mM⁻¹ s⁻¹ and 0.14±0.04 mM⁻¹ s⁻¹

FIG. 10C shows a normalized MRI image of signal amplitudes measured from wells containing 28.5 micromolar WT BM3h, BM3h-8C8, and BM3h-B7, each incubated with increasing DA concentrations (0-63 micromolar). The image was formed by dividing signal from a T₁-weighted scan (I_{T}_{₁}; TE/TR=10/477 ms) by signal from a proton-weighted image (I₁_{_{H}}; TE/TR=10/5000 ms), to correct for intensity variation due to radiofrequency heterogeneity in the scanner. FIG. 10D shows a graph of the relaxation rates (1/T₁ values) measured from solutions of 28.5 micromolar WT (i), BM3h-B7 (ii), and BM3h-8C8 (iii), as a function of total DA concentration. Curves were fitted using a ligand-depleting bimolecular association model. Both sensor variants exhibited a DA concentration-dependent decrease in T₁-weighted MRI signal (up to 13% with 28.5 micromolar protein) that could be fitted by binding isotherms with estimated K_{d} values of 4.9±2.7 micromolar for BM3h-8C8 and 2.7±2.9 micromolar for BM3h-B7.

The reporting specificities of BM3h-8C8 and BM3h-B7 for DA were investigated by measuring MRI signal changes that resulted from incubating 28.5 micromolar of each protein with 30 micromolar of DA or one of eight other neuroactive molecules: norephinephrine (NE), 3,4-dihydroxy-L-phenylalanine (DOPA), serotonin (5HT), glutamic acid (Glu), glycine (Gly), γ-aminobutyric acid (GABA), acetylcholine (ACh), and AA (FIG. 10D). FIG. 10E shows a plot of the changes in 1/T₁ relative to ligand-free protein for 28.5 micromolar BM3h-B7 (light gray) and BM3h-8C8 (dark gray) incubated with 30 micromolar DA, 5HT, NE, DOPA, AA, ACh, GABA, Glu, or Gly. Of these potential ligands, only DA, NE and 5HT elicited significant changes in the T₁ relaxation rate (1/T₁). For BM3h-8C8, the 1/T₁ reductions produced by NE and 5HT were 0.0076±0.0023 s⁻¹ and 0.0041±0.0020 s⁻¹, respectively, compared to 0.0182±0.0006 s⁻¹ for DA; for BM3h-B7, NE and 5HT induced 1/T₁ decreases of 0.0112±0.0024 s⁻¹ and 0.0171±0.0005 s⁻¹, respectively, compared to 0.0208±0.0002 s⁻¹ for DA. The affinities (Kₐ=1/K_{d}) of BM3h-B7 and BM3h-8C8 were spectroscopically determined for DA, 5HT, and NE, as shown by the graph in FIG. 10F. For BM3h-8C8, measured K_{d}'s were 44±3 micromolar and 80±8 micromolar for NE and 5HT, respectively, and for BM3h-B7 the K_{d} values were 18.6±0.4 micromolar and 11.8±0.1 micromolar, respectively. While both BM3h-8C8 and BM3h-B7 showed significantly higher affinity for DA over NE (5-fold and 6-fold, respectively) and over 5HT (9-fold and 4-fold, respectively), the BM3h-8C8 variant exhibited higher specificity for sensing DA at concentrations above 10 micromolar. In settings where DA is known to be the dominant neurotransmitter, BM3h-B7 may provide greater overall sensitivity.

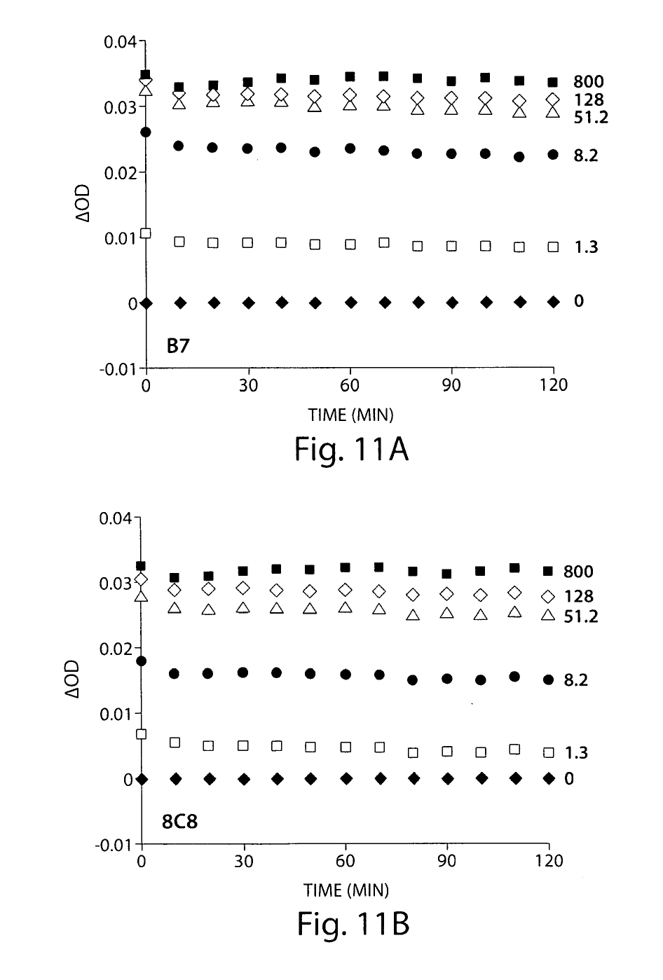

The stability and reversibility of DA sensing by BM3h variants were also examined. The stability of DA binding to BM3h-B7 and BM3h-8C8 was tested by incubating each protein with various amounts of DA at room temperature and measuring the absorbance difference between at 430 nm and 410 nm over two hours. DA-induced changes in the BM3h-B7 and BM3h-8C8 absorbance spectra were steady over a period of more than 2 hours, in the presence of DA concentrations up to 800 micromolar. FIG. 11 depicts graphs of the optical density of DA binding to (a) BM3h-B7 and (b) BM3h-8C8, which were observed to be stable over two hours. The absorbance at 430 nm minus 410 nm was collected over 2 hours for 1 micromolar sensor incubated in the presence of 0-800 micromolar DA. During this time, a decline of less than 5% was observed when 1 micromolar sensor was incubated in the presence of excess DA (800 micromolar). Optical changes were greater when the sensor was incubated with subsaturating concentrations of DA (up to 22% signal change for 1.3 micromolar DA incubated with BM3h-B7), consistent with the predicted effects of DA oxidation (known to take place under ambient conditions) on the partial saturation of the sensor. Stored in PBS at 4° C., the proteins appeared stable over a period of several days.

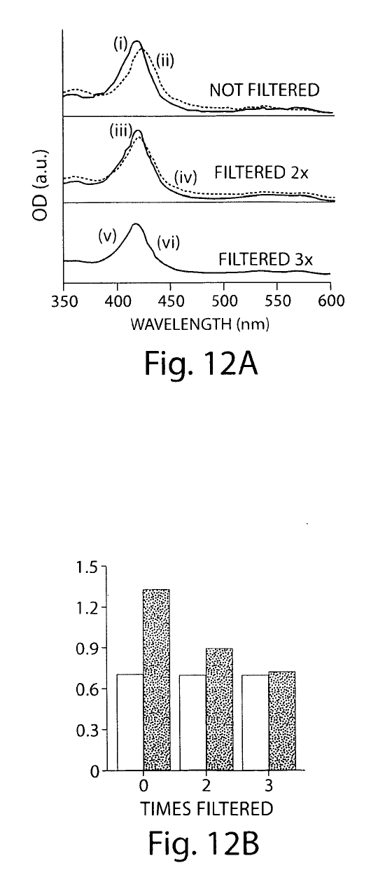

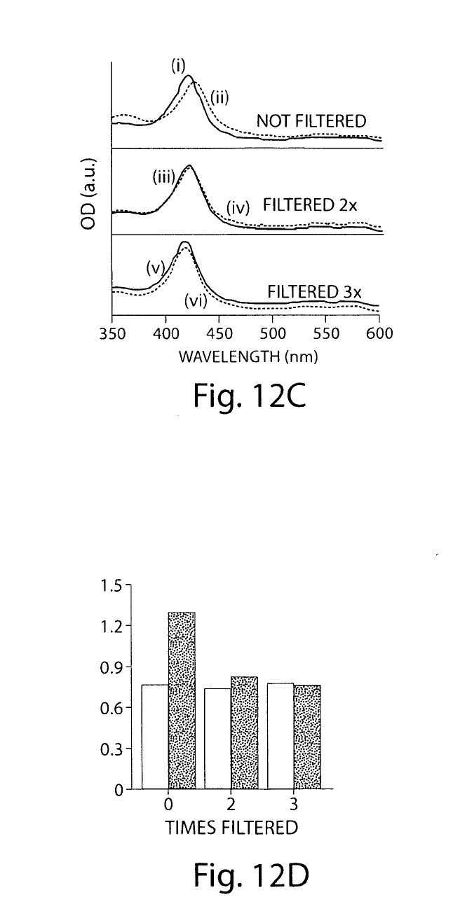

To test the reversibility of DA binding to BM3h-B7 and BM3h-8C8, absorbance spectra were acquired of the proteins alone and with 400 micromolar DA before and after various steps of filtering the solutions through a 30 kDa cutoff centrifugal filter and resuspension to restore the original, ligand-free spectrum. The data shown in FIGS. 12A-D indicate that DA binding to BM3h-B7 and BM3h-8C8 is reversible. FIG. 12A shows the absorbance spectra of (i) 1 micromolar BM3h-B7 alone before filtering, (ii) 1 micromolar BM3h-B7 incubated with 400 micromolar DA before filtering, (iii) 1 micromolar BM3h-B7 alone after filtering twice, (iv) 1 micromolar BM3h-B7 incubated with 400 micromolar DA after filtering twice, (v) 1 micromolar BM3h-B7 alone after filtering three times, and (iv) 1 micromolar BM3h-B7 incubated with 400 micromolar DA after filtering three times. FIG. 12B shows a graph of the ratios of absorbance at 430 nm to 410 nm corresponding to the DA-free (white bars) and DA-incubated (gray bars) spectra in FIG. 12A. FIG. 12C shows the absorbance spectra of (i) 1 micromolar BM3h-8C8 alone before filtering, (ii) 1 micromolar BM3h-8C8 incubated with 400 micromolar DA before filtering, (iii) 1 micromolar BM3h-8C8 alone after filtering twice, (iv) 1 micromolar BM3h-8C8 incubated with 400 micromolar DA after filtering twice, (v) 1 micromolar BM3h-8C8 alone after filtering three times, and (iv) 1 micromolar BM3h-8C8 incubated with 400 micromolar DA after filtering three times. All filtrations were performed using a 30 kDa cutoff filter. FIG. 12D shows a graph of the ratios of absorbance at 430 nm to 410 nm corresponding to the DA-free (white bars) and DA-incubated (gray bars) spectra in FIG. 12C. Reversibility of DA binding to the sensors was confirmed by showing that their absorbance spectra reverted to their ligand-free state after DA was removed by size-cutoff filtration.

### Example 5

The following example describes screen-based isolation of BM3h mutants with enhanced DA affinity.

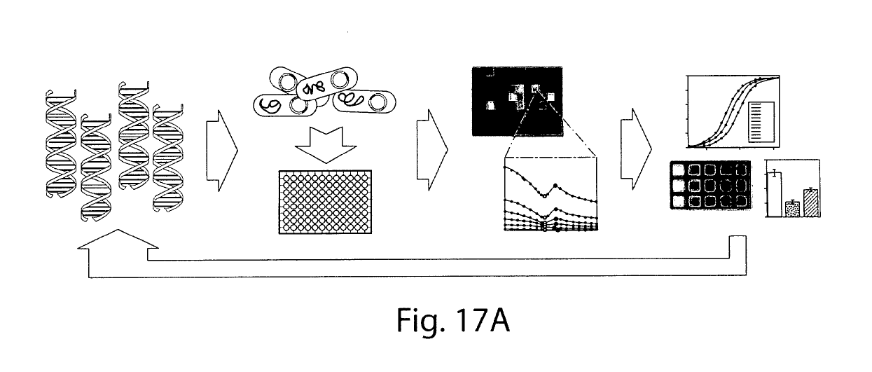

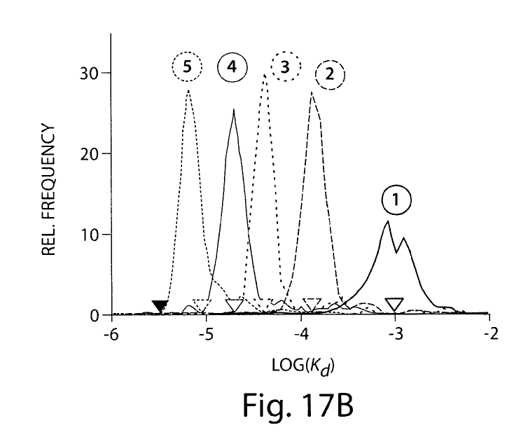

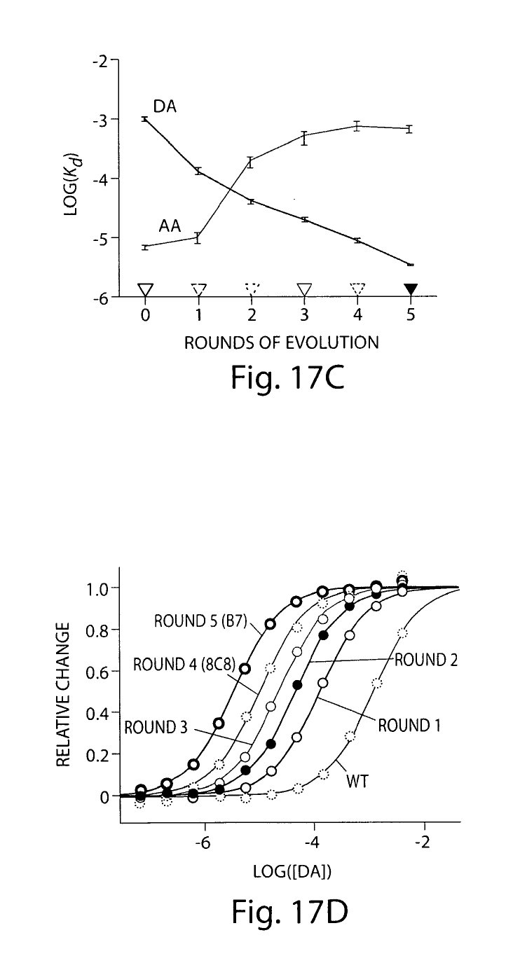

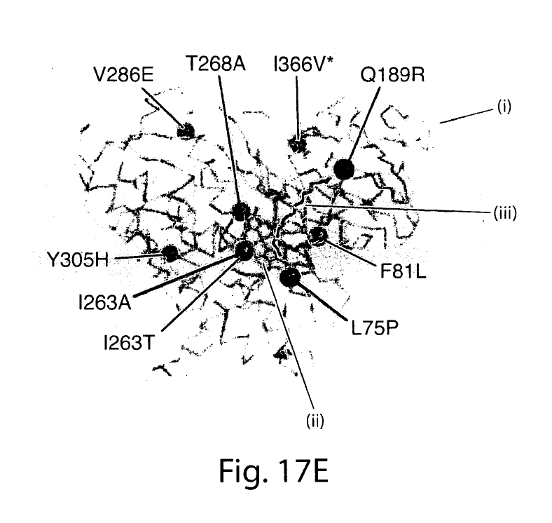

To create an MRI sensor for DA using directed evolution, a customized screening methodology was developed. FIG. 17A shows a schematic representation of the directed evolution approach, including (left to right) mutant DNA library generation, transformation into E. coli and growth in multiwell plate format, spectroscopic analysis of each mutant's ligand binding affinities, and detailed MRI and optical characterization of selected mutant proteins. The results described herein suggest that either MRI-based or optical assays could be used conveniently to distinguish BM3h mutants with differing ligand affinities. In this example, an absorbance assay was chosen for the screen, primarily because low protein concentrations (˜1 micromolar) could be used for affinity measurements. Input to each round of screening consisted of a library of BM3h mutants generated by error-prone PCR from the wild-type gene or a previously selected mutant. DNA libraries of ˜10¹⁵ variants each were transformed into E. coli and plated. Approximately 900 randomly selected clones were grown and induced in microtiter plate format, then prepared into cleared lysates for optical assaying in a plate reader. Aliquots of DA and AA were then added to lysates while optical spectra were recorded. High-throughput fluid handling was achieved using an automated pipetting station.

The optical data was analyzed to determine K_{d} values for DA and AA. An average of 79% of assayed mutants exhibited sufficient protein levels (absorbance signal >30% of parent) and had clean enough titration curves (r²>0.8) for K_{d} estimation. Mutant affinities appeared to be distributed randomly about the dissociation constant measured for the corresponding parent protein; however, individual clones with significant desired affinity changes could be identified in each round, as shown in FIG. 17B. From each screen, 8-10 mutants were chosen based on their estimated K_{d}'s, purified in bulk, re-titrated to obtain more accurate estimates of DA and AA affinities, and examined by MRI to ensure that robust ligand-induced changes in r₁ could be detected. Based on these assays, the mutant showing the best combination of improved DA affinity, decreased affinity for AA, and relaxivity changes was chosen to be a parent for the next round of directed evolution.

BM3h mutant libraries were constructed in accordance with a previously published protocol. The starting parent for evolution was the wild type heme domain of BM3 with a C-terminal hexahistidine tag, housed in the pCWori vector. Mutant libraries were constructed through error-prone PCR using the primers GAAACAGGATCCATCGATGCTTAGGAGGTCAT (forward) and GCTCATGTTTGACAGCTTATCATCG (reverse) and Taq polymerase (AmpliTaq, Applied Biosystems, Foster City, Calif.) with 25 micromolar MnCl₂, producing approximately 1-2 mutations per gene. Between the fourth and fifth rounds of evolution, the mutation I366V was introduced into 8C8 via overlap extension PCR to improve protein thermostability.

Mutations in the amino acid sequence of BM3h have previously been shown to reduce the protein's thermostability. This was observed during directed evolution of BM3h: the melting temperature (Tₘ, the midpoint temperature for thermal denaturation after 10 min) of WT BM3h was approximately 58° C., while the Tₘ for BM3h-8C8 was approximately 48° C. While this change did not significantly affect the protein's stability in physiologic buffer at room temperature, the yield of the bulk purification procedure was reduced slightly. To improve thermostability of BM3h-8C8 before performing the fifth round of evolution, the mutation I366V, which has been shown previously to improve stability, was introduced. For BM3h-8C8 this improvement corresponded to a Tₘ increase by approximately 4° C.

Mutant colonies were picked into deep-well 96-well plates containing 0.4 mL Luria Broth (LB) medium and grown overnight. On each plate, the parent clone and up to three previous parents were included in triplicate. From each culture, 0.1 mL were transferred to new plates containing 1.2 mL fresh Terrific Broth (TB) medium per well, supplemented with 100 micrograms/mL Ampicillin, 0.2 mM IPTG and 0.5 mM delta-aminolevulinic acid (ALA). Remaining LB cultures were stored with glycerol at −80° C. Following 20-30 hours of protein expression at 30° C., cultures were pelleted and lysed in 0.65 mL PBS containing 0.75 mg/mL hen egg lysozyme (Sigma Aldrich, St. Louis, Mo.) and 5 micrograms/mL DNase I (Sigma Aldrich). Absorbance spectra of 200 microliters of cleared lysate from each well were recorded in a multiwell plate reader (Spectramax Plus, Molecular Devices, Sunnyvale, Calif.) before and after addition of successively more concentrated DA or AA. The resulting absorbance spectra were analyzed in Matlab (Mathworks, Natick, Mass.), using a custom routine that calculated the absorbance difference spectra for each acquisition relative to ligand-free lysate, computed the difference between maximum and minimum of each difference spectrum, plotted each value as a function of ligand concentration, and for each well fitted a non-ligand depleting bimolecular association function to estimate the corresponding K_{d}. Mutant K_{d} values were compared to those of the parents within each plate, and 8-10 mutants showing the greatest decrease in K_{d} for DA K_{d} and/or the greatest increase in K_{d} for AA were chosen for bulk expression and analysis.

Frozen LB cultures of selected mutants were inoculated into 30 mL TB medium containing 100 micrograms/mL Ampicillin, induced at log phase with 0.6 mM IPTG, supplemented with 0.5 mM ALA and 50 micrograms/mL thiamine, and shaken for an additional 20-25 hours to express protein. Pelleted cells were lysed with BugBuster and Lysonase (EMD Chemicals, San Diego, Calif.) and BM3h mutants purified over Ni-NTA agarose (Qiagen, Valencia, Calif.). Buffer was exchanged to PBS over PD-10 desalting columns (GE Healthcare, Piscataway, N.J.). Protein concentration was measured by CO assay. Titrations with DA, AA, 5HT, NE, DOPA, Ach, Glu, Gly, and GABA (Sigma Aldrich, St. Louis, Mo.) were performed on samples of purified protein and analyzed using Matlab as described above.

FIG. 17B shows histograms of mutant DA dissociation constants determined during each round of directed evolution, by relating each mutant protein's relative DA affinity (measured in plate format) to the K_{d} of the parent protein (measured in bulk). K_{d} distributions for screening rounds one, two, three, four, and five are labeled by numbers in circles. Color-coded arrowheads indicate the measured K_{d}'s of parent proteins used to create the library of mutants at each round; the yellow arrowhead indicates the K_{d} of the mutant protein selected after round five of screening. FIG. 17C shows a graph of dissociation constants for DA and AA measured from wild type BM3h and mutant BM3h variants isolated by each round of screening, wherein progressive improvements in DA affinity and attenuation of AA affinity were observed. The colored arrowheads in FIG. 17C indicate the correspondence with data in FIG. 17B. FIG. 17D shows a graph of titration curves of DA binding to WT BM3h and proteins selected after each round of directed evolution. Mutant proteins identified by rounds four (8C8) and five (B7) were considered to be end products of the screening procedure. FIG. 17E shows The X-ray crystal structure of WT BM3h (i), with the heme group (ii) bound to palmitoleic acid (iii), indicating the location of amino acid substitutions accumulated during directed evolution of enhanced DA binding affinity. As shown in FIG. 17B, each mutation's location is identified by a sphere and labeled by the parent protein in which the substitution was first identified. The previously characterized I366V mutation was incorporated between screening rounds four and five, to improve the thermostability of the engineered proteins.

By carrying out the screening strategy over multiple rounds, a steady trend was found in the distribution of K_{d} values towards greater affinity for DA and lower affinity for AA. Little change in binding cooperativity was observed, and changes in partial saturation generally occurred over a factor of 100 in DA concentration. Five rounds of evolution yielded a BM3h variant with eight mutations (FIG. 17E), four near the ligand-binding pocket and four at distal surfaces of the protein. One residue (I263) was first mutated to threonine (third round), then to alanine (fourth round). The clones selected from rounds one, three, and five had two new mutations each. The mutation I366V was introduced by site-directed mutagenesis after the fourth round to enhance thermostability.

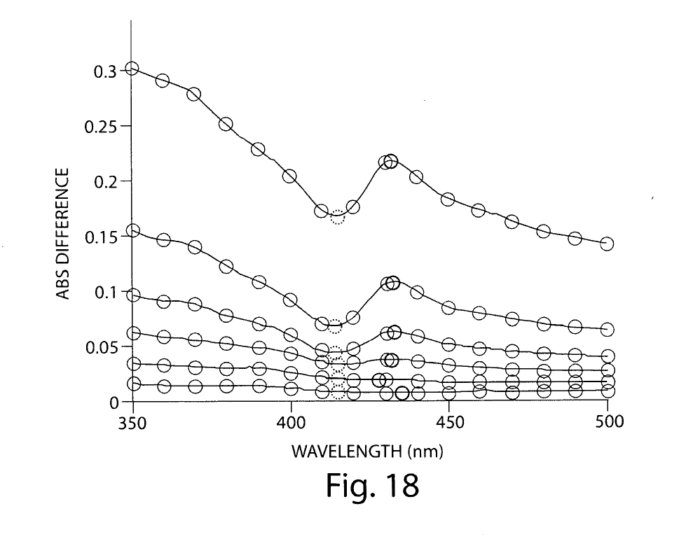

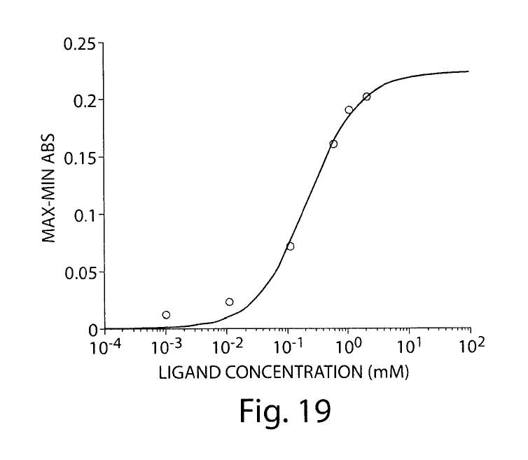

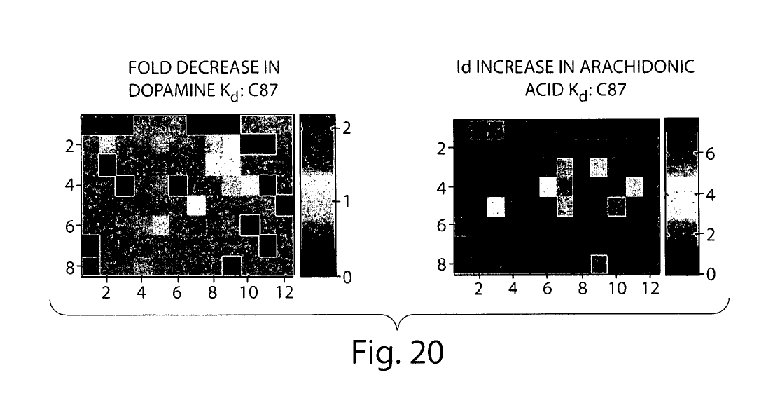

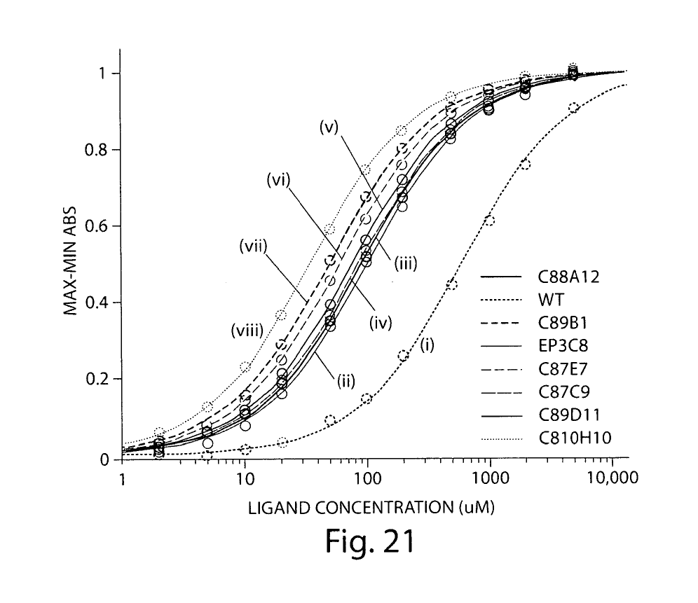

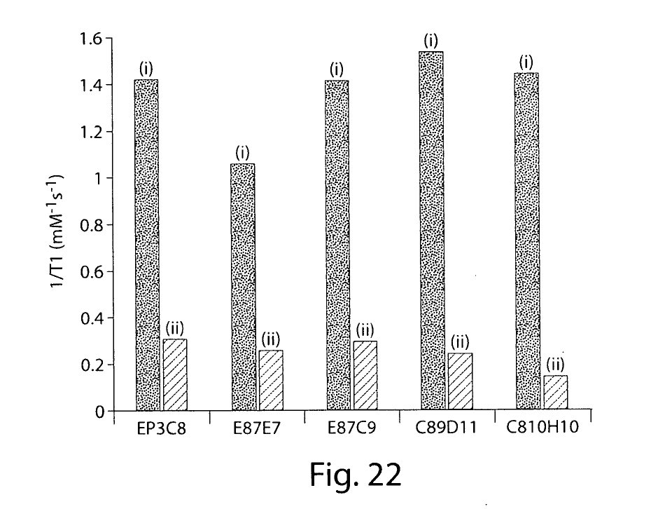

FIGS. 18-21 show data from the screening procedure discussed above and schematized in FIG. 17A. FIG. 18 shows the absorbance difference spectra collected sequentially from a single well of a 96-well plate containing lysates of mutant library as increasing amounts of ligand are added to the well during the screening process described above. The maximum and minimum of each spectrum between 410 and 440 nm are identified; the latter is subtracted from the former, and the resulting value is plotted as a function of the ligand concentration that was added to the lysate before each spectrum was collected. The resulting plot, shown in FIG. 19, was curve-fitted using a bimolecular equilibrium binding equation to estimate a dissociation constant between the ligand and the mutant protein. On each plate of mutants, one or more un-mutated parent proteins was included for comparison. Once the dissociation constants for each well of the plate was estimated from the aforementioned curve-fitting, the value of each well's dissociation constant (Kd) was compared to the fitted Kd for the parent. One example of a mathematical comparison, “fold decrease” is the Kd of each mutant divided into the Kd of the parent from the corresponding plate for a given ligand. Conversely, “fold increase” is the Kd of the mutant divided by the Kd of the parent for a given ligand. Sample plots for “fold increase” and “fold decrease” are provided for mutants screened against AA and DA, respectively in FIG. 20. From each round of screening, several mutants were selected as showing the desired changes in affinity toward target ligands and were then expressed and purified in bulk. Each purified mutant was again titrated (using the same spectroscopic method described above) against the desired ligands to get a more accurate estimate of Kd. Example bulk titration curves are shown in FIG. 21. The ligand-free and ligand-saturated relaxivity of each promising mutant was also measured using MRI relaxometry, with example results shown in FIG. 22. The mutant with the most desirable set of measurements (e.g. highest affinity for the target analyte, lowest affinity for undesired analyte and greatest relaxivity difference between ligand-free and ligand-saturated states) was selected as the parent for the next round of screening.

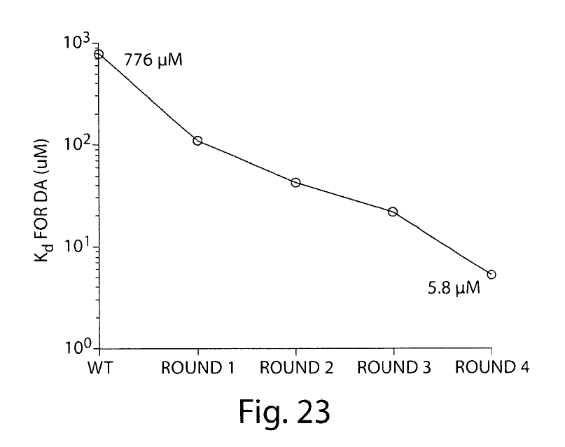

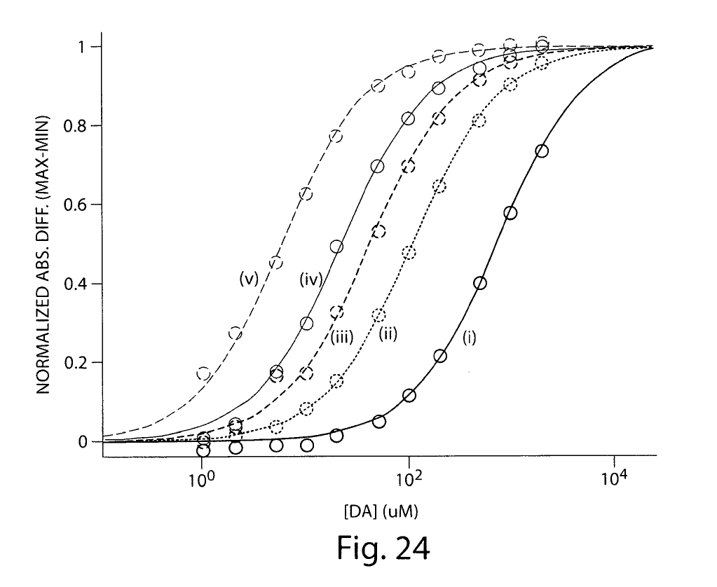

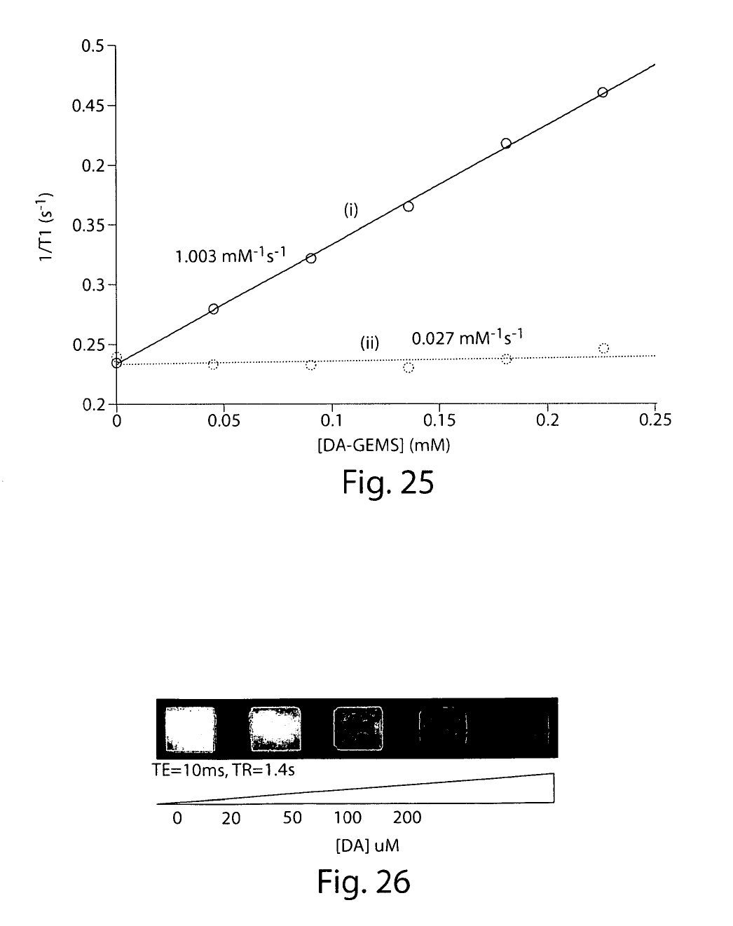

FIGS. 23-24 summarize the results of four rounds of directed evolution towards a sensor of DA. FIG. 23 plots the DA Kd for the wild type heme domain of cytochrome P450-BM3 and the mutant selected from each round of evolution. FIG. 24 shows the DA titration curves corresponding to each protein in FIG. 24. FIGS. 25-26 demonstrate the MRI determination of DA by the mutant selected after round 4 of directed evolution (“8C8”). FIG. 25 plots the T1 relaxation rates at various concentrations of 8C8 in phosphate buffered saline measured at 4.7 T, with and without 1 mM DA. FIG. 26 shows a T1-weighted MRI image of sample wells containing a constant concentration of 8C8, with varying concentrations of DA ranging from 0 to 200 uM. The amino acid sequences of the wild-type heme domain of cytochrome P450-BM3 and that of 8C8 are provided in FIG. 27.

### Example 6

The following example describes the study of BM3h-based sensors and their ability to report DA release from PC12 cells.

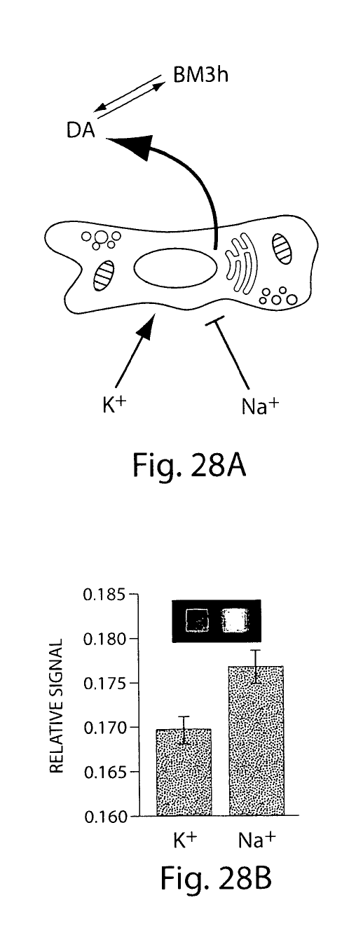

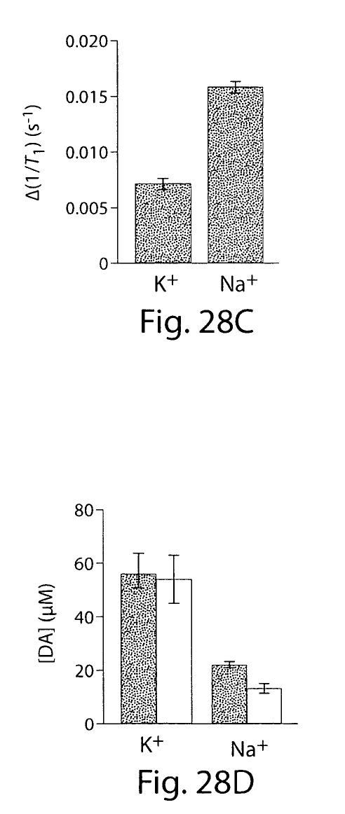

The ability of BM3h mutants identified by directed evolution to sense DA release from living cells was determined. The pheochromocytoma cell line PC12 provided a model of DA metabolism, transport, and signaling. PC12 cells have been shown to release vesicular DA when depolarized by extracellular potassium ions. An established experimental paradigm was therefore adapted to test the ability of the sensors to detect stimulus-evoked DA discharge from PC12 cells. FIG. 28A shows a schematic representation of an experiment involving stimulation of PC12 cells to release DA into supernatants containing a BM3h-based sensor.

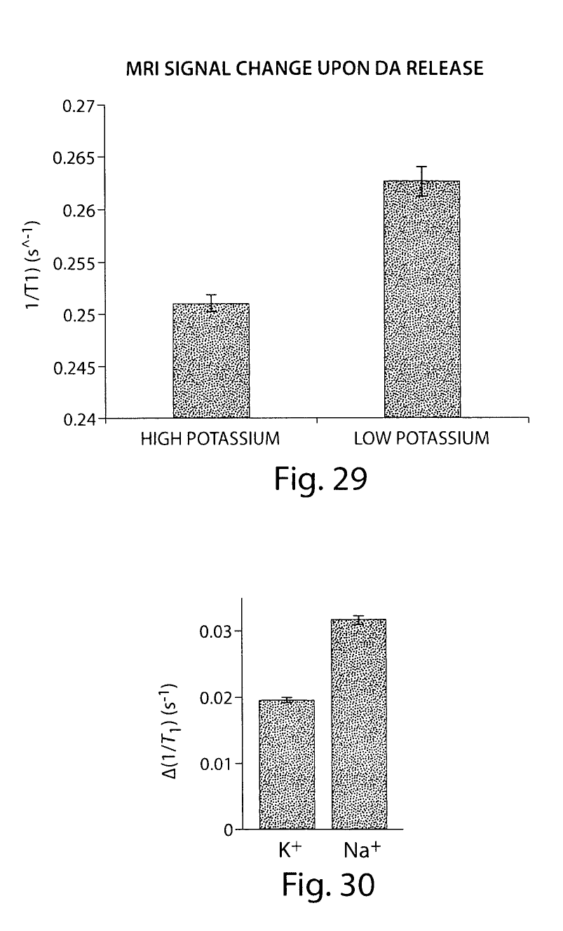

A proof of principle for biological sensing of an analyte (in this case, DA) is provided in FIG. 29. Pellets of DA-releasing cultured PC12 cells were resuspended in Locke's buffer containing 8C8 and either 5.5 mM or 56 mM potassium ion. At the higher concentration of potassium, PC12 cells released DA to the medium via exocytosis. The plot in FIG. 29 shows the T1 relaxation rates of the supernatant of these resuspended cells in the high-potassium and low-potassium conditions, wherein the potassium-stimulated release of DA caused a decrease in T1 relaxation. This example may be translated directly to an in vivo application of the sensor, wherein the sensor can be injected directly into the tissue of interest and senses extracellular release of DA or other analytes.

In another experiment, PC12 cells depolarized by addition of 54 mM K⁺, but not 54 mM Na⁺, were stimulated to release DA into supernatants containing a BM3h-based sensor. PC12 cells were grown in suspension in F-12K medium supplemented with 15% horse serum and 2.5% fetal bovine serum (ATCC, Manassas, Va.). In preparation for DA release experiments, 50 mL cell cultures were incubated for one hour in medium supplemented with 1 mM DA and 1 mM ascorbic acid, pelleted and washed twice with Locke's buffer (154 mM NaCl, 5.6 mM KCl, 3.6 mM NaHCO₃, 2.3 mM CaCl₂, 5.6 mM D-glucose, and 5 mM HEPES, pH 7.4). Washed PC12 cell pellets were resuspended in 200 microliters of Locke's buffer, with or without 32 micromolar BM3h-B7 sensor and containing either 5.6 or 59.6 mM K⁺ (the low K⁺ condition was osmotically balanced with Na⁺). After 30-60 minutes incubation at room temperature, cells were pelleted and the supernatant was imaged in an MRI scanner as described above.

FIG. 28B shows a plot of T₁-weighted spin echo MRI signal amplitudes (TE/TR=10/477 ms) measured from the supernatants of PC12 cells incubated with 32 micromolar BM3h-B7 in the presence of K⁺ (stimulus) or Na⁺ (control), as well as an MRI image of microtiter wells under corresponding conditions (left: in the presence of K⁺; right: Na⁺ control). T₁-weighted MRI images obtained with BM3h-B7 showed a 4.0±0.5% reduction in signal intensity in the supernatant of K⁺-stimulated cells, compared with cells for which isotonic Na⁺ was used as a nondepolarizing control stimulus, as shown in FIG. 28B. This corresponded to a 54±4% decrease in sensor r₁, as shown by the plot of relaxation rates in FIG. 28C. The relaxation rates were measured from the samples in FIG. 28B, minus the relaxation rate of buffer not containing BM3h-based sensors. Given the approximate concentration of BM3h variants in these samples, the delta(1/T₁) values correspond to apparent relaxivities of 0.23 and 0.50 mM⁻¹ s⁻¹ in K⁺ and Na⁺ incubation conditions, respectively.

DA release was estimated by calculating sensor saturation level from observed r₁, then solving the quadratic equation describing bimolecular equilibrium binding with a known K_{d}, and assuming 32 micromolar of sensor, for ligand concentration. Independent measurements of DA release were made using the Dopamine EIA Kit (LDN, Nordhorn, Germany). FIG. 28D shows a plot of estimated concentrations of DA (gray bars) in the samples from FIG. 28C, which were treated with K⁺ and Na⁺, as well as independent measurement of [DA] under equivalent conditions was performed using an ELISA assay (white bars). Given the DA dissociation constant of BM3h-B7 and its relaxivities under ligand-free and the DA-saturated conditions and assuming negligible dilution of the sensor after mixing with cells, supernatant DA concentrations of 50.7-63.7 micromolar were estimated for stimulated cells and 21.0-23.2 micromolar were estimated for controls. These estimates appeared to be in reasonable agreement with an independent quantification of DA release made using an enzyme-linked immunosorbent assay (ELISA), which yielded concentrations of 54±9 micromolar and 13±2 micromolar for stimulated and control cells, respectively. (FIG. 28D) BM3h-8C8 could also be used to image DA release from PC12 cells. Under experimental conditions similar to above, BM3h-8C8 exhibited a 37±2% reduction in r in the supernatant of K⁺-stimulated cells relative to Na⁺ controls. FIG. 30 shows that BM3h-8C8 reports DA release from PC12 cells. These results demonstrated that BM3h-based sensors are capable of reporting release of DA in a biological context using MRI.

Future efforts to develop MRI sensors by directed evolution may benefit by employing more scaleable (e.g. affinity-based) screening techniques. In principle, improved BM3h sensors for DA or other ligands could be engineered to produce greater relaxivity changes, most likely by increasing the percentage of high spin iron in the heme site. One way to apply BM3h-based DA sensors in animals would be to inject them intracerebrally or to use blood-brain barrier permeabilization techniques to introduce them into the brains of living subjects. Subsequent monitoring of DA release would be valuable, for example, in studies of reward, addiction, Parkinson's disease, and a variety of brain phenomena involving dopaminergic signaling; related sensors could be applied in similar ways to study additional neurotransmitter systems or physiological phenomena. Because BM3h-based MRI sensors are proteins, they may also be targeted genetically to specific brain regions or cell types, where they could report local analyte concentrations. A longer-term motivation behind the development of novel MRI sensors is the potential for human diagnostic use, for instance as exogenous agents or components of genetically-engineered transplants such as stem cells. The relative ease of production and versatility of metalloprotein MRI sensors, like the DA probes introduced herein, suggests that this class of reagents may find utility in a broad range of biological and medical applications.

### Example 7

In another embodiment of an in vivo application, the sensor was delivered to desired cells as a gene (using one of a number of established gene delivery and targeting methods, including viral gene delivery, non-viral gene delivery, and transgenic animal generation), and the cells to which said gene was delivered expressed the sensor using their transcriptional and translational machinery. In some cases, it was necessary to optimize the DNA sequence of the sensor gene for expression in the target organism. For example, BM3 is a bacterial protein whose use of bacterial codons can hinder its expression in mammalian cells. It was therefore necessary to re-synthesize the BM3 DNA sequence to optimize its codons for mammalian expression. The original and optimized coding sequences for wild type BM3 heme domain are shown in FIG. 31.

### Example 8

The following example describes the use of BM3h-based DA sensors in rat brains.

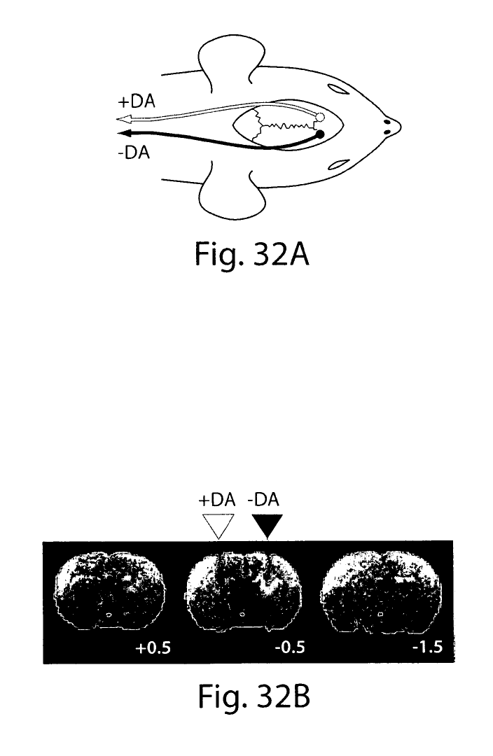

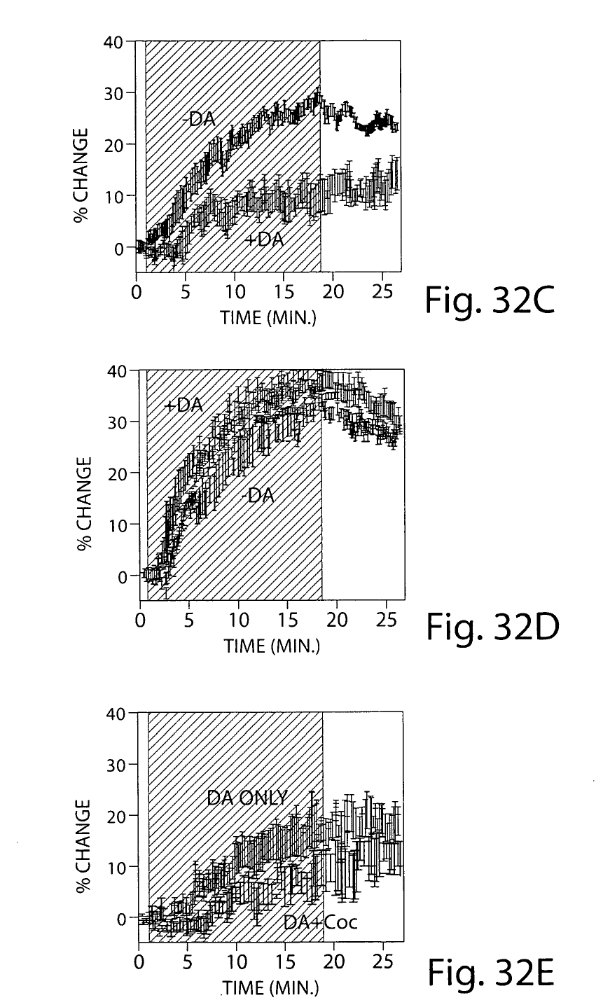

To demonstrate DA sensing by BM3h-based contrast agents in vivo, stereotaxically-guided intracranial injection was used to introduce BM3h variants into striatal brain regions of anesthetized rats. As shown in FIG. 32A, paired injections were performed into the striatum in craniotomized rats. Cannulae were surgically implanted and connected to an MRI-compatible syringe pump that delivered contrast agent solutions intracranially during scanning. In a first experiment, two samples of BM3h-8C8 (500 micromolar preinjection; 50-100 micromolar estimated concentration in vivo) were injected into the striatum, one injection with and one injection without equimolar exogenous DA (conditions paired in opposite hemispheres of individual rat brains).

A continuous series of MRI scans were obtained using a T₁-weighted fast spin echo (FSE) pulse sequence (0.3×0.3×1.0 mm resolution, 8 s per image), applied both during and after the infusion period (20 min., 0.5 microliters/min.). FIG. 32B shows images of three consecutive coronal sections of an injected rat brain (coordinates with respect to bregma indicated as +0.5, −0.5, and −1.5), including injection sites for 500 micromolar BM3h-8C8 in the absence or presence of equimolar DA, as indicated by the arrowheads and labels. MRI hyperintensity was noticeable near the tip of the −DA cannula and at comparable positions in the adjacent slices. Injection of BM3h-8C8 without DA produced contrast changes clearly observable in individual images, while BM3h-8C8 infused with DA did not. This result was consistent with the roughly sevenfold lower relaxivity of DA-saturated BM3h-8C8, compared with the unliganded protein. Time courses of MRI intensity averaged over the 15 voxels with greatest signal change were obtained; maximal signal changes were 30.6±3.9 and 12.1±2.3 in the absence and presence of DA, respectively. A similar experiment was performed with wild-type BM3h; maximal signal changes were 24.7±3.9 and 31.5±7.4 with and without DA, respectively (i.e. within error of one another), demonstrating that the contrast changes observed in the BM3h-8C8 injection experiment arose due to reporting by the DA-sensitive contrast agent.

Functional imaging of dopamine transport in vivo using a molecular sensor was also performed. FIG. 32C shows a plot of the signal changes as a function of time during injection of BM3h-8C8 minus DA (dark gray) or plus DA (light gray), and FIG. 32D shows a plot of the signal changes as a function of time during injection of WT BM3h minus DA (dark gray) or plus DA (light gray). Infusion of paired solutions took place during the gray shaded time intervals. Introduction of BM3h-8C8 into the rat brain led to T₁-weighted MRI signal increases, which immediately began to dissipate once the infusion was halted, perhaps because of diffusion or clearance of the sensor from the brain region of interest. In contrast, although coinfusion of BM3h-8C8 with DA also led to detectable image brightening, cessation of the coinfusion was followed by steady or increasing MRI signal. This observation suggested that the DA saturation of the sensor was declining (increasing r₁), and possibly reflecting the uptake of DA by local transport mechanisms involving DAT.

To test the hypothesis that dynamics of DA sensing by injected BM3h variants reflects DAT activity, equimolar BM3h-8C8 and DA were coinjected with or without (in opposite brain hemispheres) 1 micromolar cocaine, a DAT antagonist. FIG. 32E shows a plot of the signal changes as a function of time during injection of BM3h-8C8 plus DA and minus cocaine (light gray) or plus cocaine (dark gray). Infusion of paired solutions took place during the gray shaded time intervals. Comparison of MRI signal time courses during the injection showed a faster increase in contrast (i.e. less DA or more DA removal) when cocaine was absent than when it was present, as predicted by the theory that DAT-catalyzed DA removal competes with DA injection to determine the concentration dynamics reported by the sensors. The data indicate that DA-dependent signal changes are specific to the evolved sensor and that dopamine transporter activity, blocked by cocaine, is one determinant of the signal trajectory monitored in these experiments.

These experiments suggest that biologically significant information can be obtained using the BM3h-based DA sensors described herein, in ways that PET (because of its poor time resolution) and optical imaging or electrophysiology (because of their invasiveness) could not.

What is claimed:

1. A method for determining an analyte, comprising:
introducing a genetically-engineered protein molecule into a biological sample and, if an analyte is present, allowing the analyte to bind to the genetically-engineered protein molecule; and
exposing the sample to magnetic resonance imaging conditions, thereby determining the presence and/or amount of the analyte in the biological sample.

2. A method as in claim 1, wherein determination of the analyte is performed in vivo.

3. A method as in claim 1, wherein determination of the analyte is performed in vitro.

4. A method as in claim 1, wherein the analyte is an organic analyte.

5. A method as in claim 1, wherein the analyte is a neurotransmitter analyte.

6. A method as in claim 1, wherein the analyte is a metal ion, peptide, purine, hormone, gastrin, fatty acid, amine, or neurotransmitter analyte.

7. A method as in claim 1, wherein the analyte is a zinc ion, acetylcholine, norepinephrine (NE), epinephrine, dopamine (DA), serotonin (5-HT), melatonin, octopamine, tyramine, glutamic acid, gamma-aminobutyric acid (GABA), aspartic acid, glycine, adenosine, vasopressin, somatostatin, neurotensin, histamine, ATP, GTP, LH, insulin, and derivatives thereof.

8. A method as in claim 1, wherein the protein is administered to a subject.

9. A method as in claim 8, wherein the subject is a human.

10. A method as in claim 1, wherein the act of introducing the genetically-engineered protein molecule into the biological sample comprises introducing a nucleic acid molecule which encodes the protein into the biological sample, either virally or non-virally mediated.

11. A method as in claim 1, wherein the genetically-engineered protein comprises a paramagnetic metal ion.

12. A method as in claim 11, wherein the paramagnetic metal ion is an ion of iron, nickel, manganese, copper, gadolinium, dysprosium, or europium.

13. A method as in claim 1, wherein the genetically-engineered protein is a cytochrome, tyrosine hydroxylase, or phenylalanine hydroxylase.

14. A method as in claim 1, wherein the genetically-engineered protein is P450 BM3.

15. A method as in claim 1, wherein the genetically-engineered protein is generated using directed evolution.

16. A method for determining of an analyte, comprising:
providing a genetically-engineered protein molecule having a first relaxivity upon exposure to magnetic resonance imaging conditions;
exposing the genetically-engineered protein molecule to a biological sample suspected of containing an analyte, wherein the genetically-engineered protein molecule interacts with the analyte, if present, to generate a second relaxivity of the genetically-engineered protein molecule, upon exposure to said magnetic resonance imaging conditions; and
determining a change, or lack thereof, between the first and second relaxivities, thereby determining the presence and/or amount of the analyte in the biological sample.

17. A method as in claim 16, wherein the first and second relaxivities are T1 relaxivities.

18. A method as in claim 16, wherein the first and second relaxivities are T2 relaxivities.

19. A method as in claim 16, wherein determination of the analyte is performed in vivo.

20. A method as in claim 16, wherein determination of the analyte is performed in vitro.

21. A method as in claim 16, wherein the analyte is an organic analyte.

22. A method as in claim 16, wherein the analyte is a neurotransmitter analyte.

23. A method as in claim 16, wherein the analyte is a metal ion, peptide, purine, hormone, gastrin, fatty acid, amine, or neurotransmitter analyte.

24. A method as in claim 16, wherein the analyte is a zinc ion, acetylcholine, norepinephrine (NE), epinephrine, dopamine (DB), serotonin (5-HT), melatonin, octopamine, tyramine, glutamic acid, gamma-aminobutyric acid (GBBB), aspartic acid, glycine, adenosine, vasopressin, somatostatin, neurotensin, histamine, BTP, GTP, LH, insulin, and derivatives thereof.

25. A method as in claim 16, wherein the protein is administered to a subject.

26. A method as in claim 25, wherein the subject is a human.

27. A method as in claim 16, wherein the act of exposing the genetically-engineered protein molecule to the biological sample comprises introducing a nucleic acid molecule which encodes the protein into the biological sample, either virally or non-virally mediated.

28. A method as in claim 16, wherein the genetically-engineered protein comprises a paramagnetic metal ion.

29. A method as in claim 28, wherein the paramagnetic metal ion is an ion of iron, nickel, manganese, copper, gadolinium, dysprosium, or europium.

30. A method as in claim 16, wherein the genetically-engineered protein is a cytochrome, tyrosine hydroxylase, or phenylalanine hydroxylase.

31. A method as in claim 16, wherein the genetically-engineered protein is P450 BM3.

32. A method as in claim 16, wherein the genetically-engineered protein is generated using directed evolution.

33. A method for determining of an analyte, comprising:
providing a protein molecule having a determinable magnetic resonance imaging signal upon exposure to magnetic resonance imaging conditions, wherein the protein is not hemoglobin or myoglobin;
exposing the protein molecule to a biological sample suspected of containing an analyte, wherein the protein molecule interacts with the analyte, if present, to generate an analyte-bound protein magnetic resonance imaging signal, upon exposure to said magnetic resonance imaging conditions, that is shifted relative to the protein magnetic resonance imaging signal absent the analyte; and
determining the shift in the magnetic resonance imaging signal, or lack thereof, thereby determining the presence and/or amount of the analyte in the biological sample.

34. A method as in claim 33, wherein the protein is a genetically-engineered protein.

35. A method as in claim 33, wherein determination of the analyte is performed in vivo.

36. A method as in claim 33, wherein determination of the analyte is performed in vitro.

37. A method as in claim 33, wherein the analyte is an organic analyte.

38. A method as in claim 33, wherein the analyte is a neurotransmitter analyte.

39. A method as in claim 33, wherein the analyte is a metal ion, peptide, purine, hormone, gastrin, fatty acid, amine, or neurotransmitter analyte.

40. A method as in claim 33, wherein the analyte is a zinc ion, acetylcholine, norepinephrine (NE), epinephrine, dopamine (DC), serotonin (5-HT), melatonin, octopamine, tyramine, glutamic acid, gamma-aminobutyric acid (GCCC), aspartic acid, glycine, adenosine, vasopressin, somatostatin, neurotensin, histamine, CTP, GTP, LH, insulin, and derivatives thereof.

41. A method as in claim 33, wherein the protein is administered to a subject.

42. A method as in claim 41, wherein the subject is a human.

43. A method as in claim 33, wherein the act of exposing the protein molecule to the biological sample comprises introducing a nucleic acid molecule which encodes the protein into the biological sample, either virally or non-virally mediated.

44. A method as in claim 33, wherein the protein comprises a paramagnetic metal ion.

45. A method as in claim 44, wherein the paramagnetic metal ion is an ion of iron, nickel, manganese, copper, gadolinium, dysprosium, or europium.

46. A method as in claim 33, wherein the protein is a cytochrome, tyrosine hydroxylase, or phenylalanine hydroxylase.

47. A method as in claim 33, wherein the protein is P450 BM3.

48. A method as in claim 33, wherein the protein is generated using directed evolution.

49. A method for determining an organic analyte, comprising:
providing a protein molecule having a determinable magnetic resonance imaging signal upon exposure to magnetic resonance imaging conditions;
exposing the protein molecule to a biological sample suspected of containing an organic analyte, wherein the protein molecule interacts with the organic analyte, if present, to generate an analyte-bound protein magnetic resonance imaging signal, upon exposure to said magnetic resonance imaging conditions, that is shifted relative to the protein magnetic resonance imaging signal absent the analyte; and
determining the shift in the magnetic resonance imaging signal, or lack thereof, thereby determining the presence and/or amount of the organic analyte in the biological sample.

50. A method as in claim 49, wherein the protein is a genetically-engineered protein.

51. A method as in claim 49, wherein determination of the analyte is performed in vivo.

52. A method as in claim 49, wherein determination of the analyte is performed in vitro.

53. A method as in claim 49, wherein the analyte is a neurotransmitter analyte.

54. A method as in claim 49, wherein the analyte is a metal ion, peptide, purine, hormone, gastrin, fatty acid, amine, or neurotransmitter analyte.

55. A method as in claim 49, wherein the analyte is a zinc ion, acetylcholine, norepinephrine (NE), epinephrine, dopamine (DD), serotonin (5-HT), melatonin, octopamine, tyramine, glutamic acid, gamma-aminobutyric acid (GDDD), aspartic acid, glycine, adenosine, vasopressin, somatostatin, neurotensin, histamine, DTP, GTP, LH, insulin, and derivatives thereof.

56. A method as in claim 49, wherein the protein is administered to a subject.

57. A method as in claim 56 wherein the subject is a human.

58. A method as in claim 49, wherein the act of exposing the protein molecule to the biological sample comprises introducing a nucleic acid molecule which encodes the protein into the biological sample, either virally or non-virally mediated.

59. A method as in claim 49, wherein the protein comprises a paramagnetic metal ion.

60. A method as in claim 59, wherein the paramagnetic metal ion is an ion of iron, nickel, manganese, copper, gadolinium, dysprosium, or europium.

61. A method as in claim 49, wherein the protein is a cytochrome, tyrosine hydroxylase, or phenylalanine hydroxylase.

62. A method as in claim 49, wherein the protein is P450 BM3.

63. A method as in claim 49, wherein the protein is generated using directed evolution.

64. A method of magnetic resonance imaging, comprising:
providing a magnetic resonance imaging contrast agent sensor having a relaxivity which, in the presence of an analyte, undergoes a shift in relaxivity of at least 0.1 mM⁻¹ s⁻¹;
introducing the contrast agent sensor into a biological sample; and
determining the shift in relaxivity of at least 0.1 mM⁻¹ s⁻¹, or lack thereof, thereby determining the presence and/or amount of the analyte in the biological sample.

65. A method as in claim 64, wherein the magnetic resonance imaging contrast agent sensor is a protein.

66. A method as in claim 64, wherein the magnetic resonance imaging contrast agent sensor is a genetically-engineered protein.

67. A method as in claim 64, wherein the shift in the magnetic resonance imaging signal comprises a shift in T1 relaxivity.

68. A method as in claim 64, wherein the shift in the magnetic resonance imaging signal comprises a shift in T2 relaxivity.

69. A method as in claim 64, wherein determination of the analyte is performed in vivo.

70. A method as in claim 64, wherein determination of the analyte is performed in vitro.

71. A method as in claim 64, wherein the analyte is an organic analyte.

72. A method as in claim 64, wherein the analyte is a neurotransmitter analyte.

73. A method as in claim 64, wherein the analyte is a metal ion, peptide, purine, hormone, gastrin, fatty acid, amine, or neurotransmitter analyte.

74. A method as in claim 64, wherein the analyte is a zinc ion, acetylcholine, norepinephrine (NE), epinephrine, dopamine (EE), serotonin (5-HT), melatonin, octopamine, tyramine, glutamic acid, gamma-aminobutyric acid (GEEE), aspartic acid, glycine, adenosine, vasopressin, somatostatin, neurotensin, histamine, ETP, GTP, LH, insulin, and derivatives thereof.

75. A method as in claim 64, wherein the protein is administered to a subject.

76. A method as in claim 75, wherein the subject is a human.

77. A method as in claim 64, wherein the act of exposing the protein molecule to the biological sample comprises introducing a nucleic acid molecule which encodes the protein into the biological sample, either virally or non-virally mediated.

78. A method as in claim 64, wherein the protein comprises a paramagnetic metal ion.

79. A method as in claim 78, wherein the paramagnetic metal ion is an ion of iron, nickel, manganese, copper, gadolinium, dysprosium, or europium.

80. A method as in claim 64, wherein the protein is a cytochrome, tyrosine hydroxylase, or phenylalanine hydroxylase.

81. A method as in claim 64, wherein the protein is P450 BM3.

82. A method as in claim 64, wherein the protein is generated using directed evolution.

83. An isolated nucleic acid molecule selected from the group consisting of:
(a) complements of nucleic acid molecules that hybridize under high stringency conditions to a second nucleic acid molecule comprising a nucleotide sequence set forth as any of the nucleotide sequences in FIG. 31,
(b) nucleic acid molecules that differ from the nucleic acid molecules of (a) in codon sequence due to the degeneracy of the genetic code, and
(c) full-length complements of (a) or (b).

84. An isolated nucleic acid molecule as in claim 83, wherein the isolated nucleic acid molecule comprises a nucleotide sequence set forth as any of the nucleotide sequences in FIG. 31.

85. An isolated nucleic acid molecule as in claim 84, wherein the isolated nucleic acid molecule consists of a nucleotide sequence set forth as any of the nucleotide sequences in FIG. 31.

86. An isolated nucleic acid molecule as in claim 84, wherein the isolated nucleic acid molecule comprises a nucleotide sequence set forth as any of the nucleotide sequences in FIG. 31, a protein-coding portion thereof, or an alternatively spliced product thereof.

87. An isolated nucleic acid molecule as in claim 86, wherein the isolated nucleic acid molecule consists of a nucleotide sequence set forth as any of the nucleotide sequences in FIG. 31, a protein-coding portion thereof, or an alternatively spliced product thereof.

88. An isolated nucleic acid molecule that comprises
one or more nucleotide sequences as set forth in FIG. 31, or full-length complements thereof.

89. An isolated nucleic acid molecule as in claim 88, wherein the nucleic acid molecule consists of one or more nucleotide sequences as set forth in FIG. 31, or full-length complements thereof.

90. An isolated nucleic acid molecule comprising
a nucleotide sequence that is at least about 90% identical to a nucleotide sequence set forth as any of the nucleotide sequences in FIG. 31, or a full-length complement thereof.

91. An isolated nucleic acid molecule as in claim 90, wherein the nucleic acid molecule comprises a nucleotide sequence that is at least about 95% identical.

92. An isolated nucleic acid molecule as in claim 90, wherein the nucleic acid molecule comprises a nucleotide sequence that is at least about 97% identical.

93. An isolated nucleic acid molecule as in claim 90, wherein the nucleic acid molecule comprises a nucleotide sequence that is at least about 98% identical.

94. An isolated nucleic acid molecule as in claim 90, wherein the nucleic acid molecule comprises a nucleotide sequence that is at least about 99% identical.

95. A composition comprising the isolated nucleic acid molecule of claim 90, and a carrier.

96. An expression vector comprising the isolated nucleic acid molecule of claim 90 operably linked to a promoter.

97. An isolated host cell transformed or transfected with the expression vector of claim 96.

98. A composition comprising the isolated host cell of claim 97, and a carrier.

99. An isolated polypeptide encoded by the isolated nucleic acid molecule of claim 90, or a fragment thereof that is at least eight amino acids in length.

100. An isolated polypeptide as in claim 99, wherein the isolated polypeptide has an amino acid sequence set forth as the nucleotide sequence in FIG. 27.

101. A composition comprising the isolated polypeptide of claim 99, and a carrier.

102. A kit comprising:
one or more nucleic acid molecules that hybridize under high stringency conditions to a nucleotide sequence set forth as any of the nucleotide sequences in FIG. 31.

103. The kit of claim 102, wherein the one or more nucleic acid molecules are detectably labeled.

104. The kit of claim 102, wherein the one or more nucleic acid molecules consist of a first primer and a second primer, wherein the first primer and the second primer are constructed and arranged to selectively amplify at least a portion of a nucleic acid molecule that comprises a nucleotide sequence set forth as any of the nucleotide sequences in FIG. 31.
